(19) United States
(12) Patent Application Publication
(10) Pub. No.: US 2005/0085799 A1
(43) Pub. Date: Apr. 21, 2005

(54) Emergency medical kit, respiratory pump, and face mask particularly useful therein

(76) Inventors: Oded Luria, Tel-Aviv (IL); David Luria, Tel-Aviv (IL)

Correspondence Address: Martin Moynihan;c/o ANTHONY CASTORINA, SUITE 207, 2001 JEFFERSON DAVIS HIGHWAY, ARLINGTON, VA, 22202, US

(21) Appl. No.: 11/007,279

(22) Filed: Dec. 9, 2004

Related U.S. Application Data
(63) Continuation-in-part of application No. PCT/IL03/00505, filed on Jun. 12, 2003.
(60) Provisional application No. 60/604,003, filed on Aug. 25, 2004.

(51) Int. Cl. A61B 17/00; A61M 16/00

(52) U.S. Cl. 606/1

(57) ABSTRACT

An emergency medical kit for use, particularly by a non-professional, to render emergency medical treatment to a patient, includes: a pressurized-oxygen container within a housing; a face mask within the housing for application to the face of a patient requiring cardiopulmonary resuscitation; and a respiratory pump within the housing connected to the pressurized-oxygen container so as to be driven thereby to supply oxygen to the mask for inhalation by the patient, and to discharge the exhalations of the patient via the face mask to the atmosphere. The face mask includes an inflatable seal around its circumference engageable with the face of the patient receiving the mask for sealing the interior of the mask; a pressure sensor sensing the pressure in the inflatable seal; and an indicator for indicating whether the face mask is properly applied to the face of the patient. The kit further includes a neck rest having straps for attaching the face mask thereto in contact with the patient's face when the patient's head is placed on the head rest. According to a most essential aspect of the invention there is provided an emergency, fully automatic kit, based on non-invasive means for performing all stages of the “chain of survival” (including: external defibrillation, ventilation and automatic chest compression) by a single operator.

### RELATED APPLICATIONS

The present application is a continuation-in-part of the U.S. National-Phase Application of International Patent Application PCT/IL03/00505, having an International Filing date 12 Jun. 2003, and includes subject matter of U.S. Provisional Application No. 60/604,003, filed Aug. 25, 2004, the entire of contents of which are hereby incorporated by reference, and the priority date of which is hereby claimed. This application further includes subject matter of U.S. Provisional Patent Application Nos. 60/387,586, filed 12 Jun. 2002 and 60/423,369, filed 4 Nov. 2002, the entire contents of which are also incorporated herein by reference, and the priority dates of which are also hereby claimed.

### FIELD AND BACKGROUND OF THE INVENTION

The present invention relates to an emergency non-invasive medical kit for rendering emergency medical treatment to a patient, and also to a respiratory pump and a face mask particularly useful in such a kit. The invention is particularly described below with respect to an automatic, simple to operate, emergency respiratory and defibrillator system that can be successfully operated by even a single bystander during emergency conditions and before the arrival of a professional medical team to the scene. While the described medical kit is particularly designed for use by a non-professional person, it will be appreciated that the described medical kit could also be provided in ambulances or hospitals for use by professional personnel. Apparatus constructed in accordance with the present invention may contain advanced Human Machine Interface (HMI) and a control system facilitating a medical treatment for patients of different ventilation requirements and at different medical scenarios of cardiac arrest and respiratory emergencies. The rationale behind the invention is the vital need for improving the survival rate of cardiac arrest victims. Cardiac arrest is the underlying cause of sudden death in two-thirds of out-of-hospital deaths. An early start of cardiopulmonary resuscitation and early defibrillation is vital in most cases, since a delay of even a few minutes may lessen the chances of the patient's recovery. Unfortunately, in most emergency conditions, an Intensive Care Unit (ICU) is not immediately available, and critical time may be lost. Also, more than half of the cases of cardiac arrest occur at home. Therefore it is important that a single, unprofessional rescuer can start the rescue operation. In addition, mask-ventilation is regarded as one of the most skill-demanding procedures, mostly because of the difficulty of sealing the mask to the face. When caregivers use existing ventilation masks, they usually must hold it with two hands to ensure adequate head tilt and open airways.

Since bystanders at the scene of an unexpected cardiac emergency are faced with a sudden crisis, they may feel panicky, anxious and helpless. Most people have no skills in performing basic cardiopulmonary resuscitation. Those who have some knowledge often fear catching diseases (e.g. AIDS, Hepatitis) by mouth-to-mouth ventilation (an inherent part of the CPR), or are repelled by the patient's physical characteristics (the presence of saliva, blood or emesis), or are concerned of making wrong decisions leading to further damage to the patient. As a result, early Cardiopulmonary Resuscitation (CPR) by bystanders starts in only 5-30 percent of witnessed cardiac arrest cases.

Recently, a large variety of Automatic External Defibrillator (AED) devices required for the Early Defibrillation stage of the “chain of survival” concept have become available in public places. In spite of their user-friendliness in analyzing the patient's cardiac condition, and the simplicity of activating the electric shock, mouth-to-mouth ventilation and manual chest compression are needed as an integral part of their operation. This limits the wide use of such devices by bystanders. Furthermore, even in cases where CPR starts, if ventilation is done without the supply of oxygen but with air, this may definitely reduce survival rate.

Thus, there is an urgent need for an emergency fully automatic kit based on non-invasive means which can be used by a single bystander to render vital medical treatments in emergency situations of out-of-hospital, during ambulance transportation and in-hospital conditions, for performing all stages of the “chain of survival” including external defibrillation, ventilation and automatic chest compression.

The prior art has attempted to address the need for providing non-professional persons equipment intended for emergency situations. Examples of such equipment are described in U.S. Pat. Nos. 4,197,842, 4,198,963, 4,297,990, 5,520,170, 5,782,878, 5,857,460, 5,873,361, 5,975,081, 5,979,444, 6,029,667, 6,062,219, 6,327,497, 6,351,671, 6,402,691, 6,428,483, 6,459,933, 6,488,029 and 6,544,190.

Some of the existing Continuous Positive Airway Pressure (CPAP) ventilators controllers are based on linear or non-linear electronic circuits, which represent the respiratory cycle. These models are used to derive a transfer function of circuit pressure, flow and a real time estimate of resistance, elasticity, lung compliance of the patient's respiratory system, and also to estimate the connecting tube compliance. These estimates preferably utilize non-invasive measurements of inlet flow and pressure, and also use real time closed-loop feedback systems. Examples, of prior art models and systems used for this purpose are described in U.S. Pat. Nos. 3,036,569, 5,752,509, 6,068,602, 6,142,952, 6,257,234, 6,332,463, 6,390,091 and 6,557,553.

The proper use of a respiratory face mask requires a high degree of skill and experience. A non-professional CPR operator is not able to properly use such a mask without advanced detailed guidance. Furthermore, a good mask-to-face seal has been attained in many instances only with considerable discomfort for both the patient and the rescuer. Examples of face masks described in the prior art appear in U.S. Pat. Nos. 2,254,854, 2,931,356, 4,739,755, 4,907,584, 4,971,051, 5,181,506 and 5,540,223. Nevertheless, there is still an urgent need for providing a face mask that can be -used particularly by a non-professional person but also by a professional person, e.g., in an ambulance or in the hospital, for assuring best quality of ventilation and the ability to ventilate a patient with oxygen-enriched air

An integrated system for cardiopulmonary resuscitation, including high-pressure ventilation followed by negative end expiratory pressure and mechanical chest compression, is described in U.S. Pat. No. 4,397,306. Experimental description of the above was presented at The American Journal of Cardiology, Volume 48, Page 1053 of December 1981. A further example for a direct heart massage is described in U.S. Pat. No. 3,496,932.

### OBJECTS AND BRIEF SUMMARY OF THE INVENTION

An object of the present invention is to provide an emergency medical kit enabling ex-hospital, but also in-hospital, cardiopulmonary resuscitation to be more effectively rendered to a patient, particularly by a bystander or non-professional, but also by a professional. Another object of the invention is to provide a respiratory pump which may be efficiently driven and which provides a wide degree of automatic controls. A further object of the invention is to provide a face mask particularly useful to render an emergency medical treatment to a patient.

According to one aspect of the present invention, there is provided an emergency medical kit for rendering emergency medical treatment to a patient, comprising: a housing, a pressurized-oxygen container within the housing; a face mask within the housing and removable therefrom for application to the face of a patient requiring emergency medical treatment; and a respiratory pump within the housing; the respiratory pump being connectable to the pressurized-oxygen container so as to be driven thereby to supply oxygen to the face mask for inhalation by the patient, and to discharge exhalations of the patient to the atmosphere.

According to further features in one described preferred embodiment, the respiratory pump includes a pump housing having first and second end walls at opposite ends thereof; a partition wall between the end walls; a first piston movable between the first end wall and the partition wall and defining a first chamber with the first end wall, and a second chamber with the partition wall; a second piston movable between the partition wall and the second end wall, and defining a third chamber with the partition wall, and a fourth chamber with the second end wall; and a stem coupling the first and second pistons for reciprocation together.

According to yet further features, the kit may further comprise a pulse-oximeter detector probe for application to the patient to detect the patient's pulse. The kit preferably further comprises a plurality of electrodes for application to the patient for administering electrical pulse therapy to the patient. The electrical conductors of the pulse-oximeter detector probe, as well as of the plurality of electrodes, are preferably carried by the feed tube of the mask for connection to an electrical power supply, thereby facilitating the deployment of the pulse-oximeter detector probe, as well as of the electrodes, in a quick and simple manner.

According to still further features in the described preferred embodiments, the kit may further comprises a telephone communication system for receiving remote instructions via the telephone, a GPS locator system for determining the location of the patient being treated, a data logging system for logging data inputted or generated during the operation of the system, a visual display for displaying data inputted or generated during the operation of the system, and/or an audio instruction and alarm system for receiving instructional information and/or for operating an alarm under predetermined conditions.

According to further features in another described preferred embodiment, the kit may further include a suction tube insertable into the mouth of a patient and connectable to the respiratory pump for drawing out fluids from the patient's mouth.

In another described preferred embodiment, the kit further includes an inflatable neck rest, and may also include a manual pump and/or a gas-discharge cartridge for inflating the inflatable neck rest.

According to still further features in several described preferred embodiments, the kit further includes a neck rest having a plurality of preferably inelastic straps, and the face mask includes a plurality of strap connectors, one connectable to each of the straps, for securely mounting the face mask to the patient when the patient's head is placed on the head rest. Preferably, the face mask further includes a chin alignment bar engageable with the undersurface of the patient's chin for securing the proper aligning the lower end of the face mask with the patient's chin.

In the latter described embodiments, the connectors are carried on three arms projecting in a T-formation from the mask such that two of the arms project laterally on opposite sides of the face mask to be aligned with the opposite sides of the patient's face when the mask is applied thereto, and the third of the arms projects from an end of the face mask to face the patient's forehead when the mask is applied to the patient's face.

In a still further described embodiment, the inelastic straps on the neck rest connectable to the third arm of the face mask includes a tensioning device for changing the tension of the strap connectable to the third arm.

According to another aspect of the invention, there is provided an emergency medical kit for rendering emergency medical treatment to a patient, comprising: a receptacle; a face mask within the receptacle and removable therefrom for application to the face of a patient requiring emergency medical treatment; and a neck rest within the receptacle and removable therefrom; the neck rest being configured for supporting the neck of a patient in need of medical treatment capable of performing an automatic optimization of the degree of hyperextension (head tilt) of the patient in order to facilitate minimum airway resistance during patient's ventilation.

According to a most essential aspect of the invention there is provided an emergency, fully automatic kit, based on non-invasive means for performing all stages of the “chain of survival” (including: external defibrillation, ventilation and automatic chest compression) by a single operator.

As will be described more particularly below, the invention enables the construction of portable, compact, emergency medical kits which can be used for rendering emergency medical treatments to patients in a manner requiring a minimum of professional skill and experience such as to enable even a single bystander or other non-professional person to render such emergency medical treatments if and when required.

According to still further aspects of the invention, there are provided a respiratory pump, and also a face mask, each particularly useful in the described emergency medical kit but also useful in many other applications, e.g., for administering emergency oxygen in an ambulance, in the hospital or in an aircraft, or for otherwise rendering respiratory assistance to a person whenever it may be required.

Further features and advantages of the invention will be apparent from the description below.

### BRIEF DESCRIPTION OF THE DRAWINGS

The invention is herein described, by way of example only, with reference to the accompanying drawings. With specific reference now to the drawings in detail, it is stressed that the particulars shown are by way of example and for purposes-of illustrative discussion of the preferred embodiments of the present invention only, and are presented in the cause of providing what is believed to be the most useful and readily understood description of the principles and conceptual aspects of the invention. In this regard, no attempt is made to show structural details of the invention in more detail than is necessary for a fundamental understanding of the invention, the description taken with the drawings making apparent to those skilled in the art how the several forms of the invention may be embodied in practice.

In the drawings:

FIG. 1 is a three-dimensional view illustrating one form of an emergency medical kit constructed in accordance with the present invention, with the cover of the kit being open to illustrate many of the components therein;

FIG. 2 is a block diagram illustrating the main components in the emergency medical kit of FIG. 1;

FIG. 3 is a three-dimensional view illustrating the emergency medical kit of FIGS. 1 and 2 during use for rendering an emergency medical treatment;

FIG. 4 is a front view illustrating the face mask in the emergency medical kit of FIGS. 1-3;

FIG. 5 is a three-dimensional view illustrating the pair of neck rests in the emergency medical kit of FIGS. 1-3;

FIGS. 6a, 6b and 6c illustrate three stages in the operation of the ventilator pump in the emergency medical kit of FIGS. 1-3;

FIG. 7 is a schematic diagram illustrating an equivalent circuit that may be used for modeling the control parameters of the illustrated system;

FIGS. 8a and 8b are longitudinal sectional views illustrating the end stroke positions of another ventilator pump that may be used in the emergency medical kit;

FIG. 9 is a block diagram illustrating the main components of an emergency medical kit including the ventilator pump of FIGS. 8a and 8b;

FIG. 10 illustrates another construction of face mask that may be included in the emergency medical kit;

FIG. 11 illustrates another construction of neck rest that may be included in the emergency medical kit;

FIG. 12 illustrates the face mask of FIG. 10 and the neck rest of FIG. 11 as applied to a patient in need of emergency medical treatment;

FIG. 13 illustrates the face mask of FIG. 10 within a storage container in the form of a bag for long storage and transportation;

FIG. 14 illustrates a neck rest of an inflatable construction for compact storage and transportation;

FIG. 15 is a fragmentary view illustrating an optional method of inflating the neck rest and/or the inflatable seal in the face mask;

FIG. 16 illustrates the housing of another form of emergency medical kit construction in accordance with the present invention;

FIG. 17 is a diagram illustrating the arrangement for inflating the inflatable seal in the face mask included in the medical kit of FIG. 16;

FIG. 18 is a diagram illustrating the main components in another emergency medical kit constructed in accordance with the present invention; and

FIG. 19 illustrates one manner of using the emergency medical kit of FIG. 18 for rendering emergency medical treatment to a patient.

### DESCRIPTION OF SEVERAL PREFERRED EMBODIMENTS

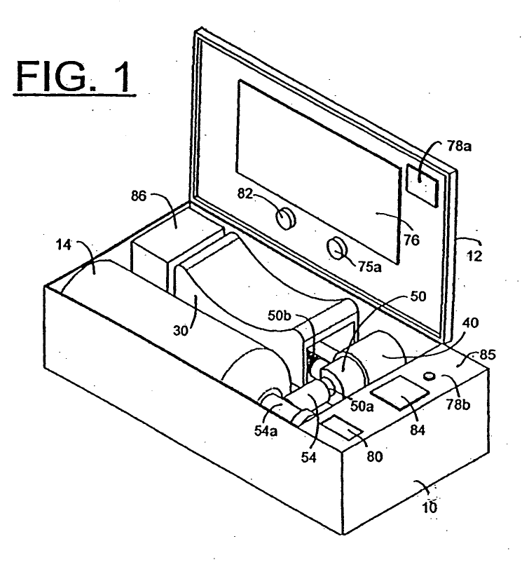

### The Embodiment of FIGS. 1-7: Overall Construction

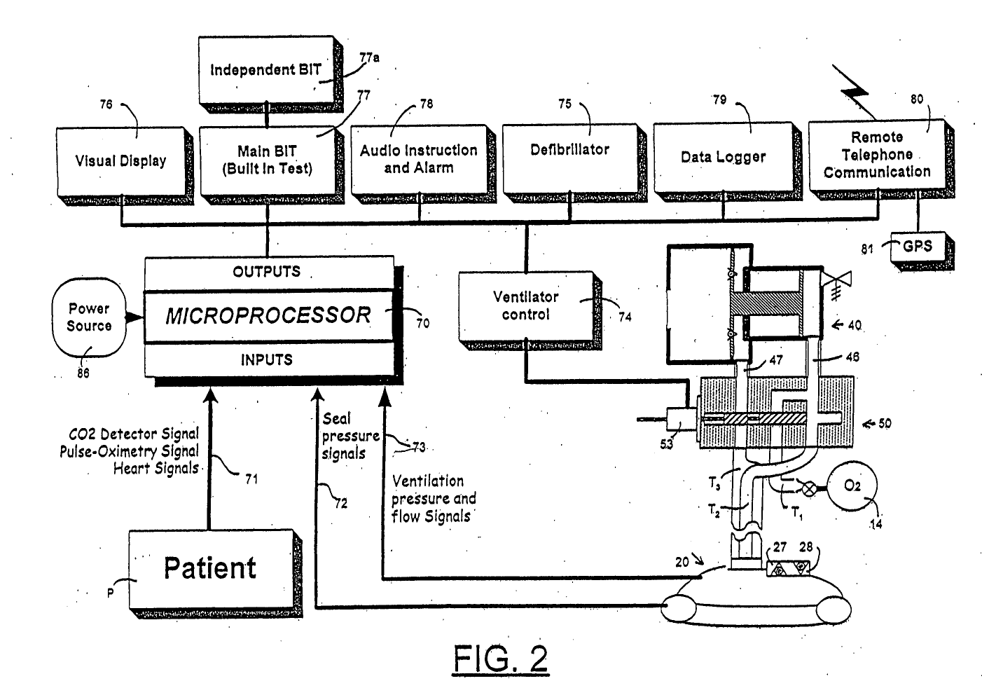

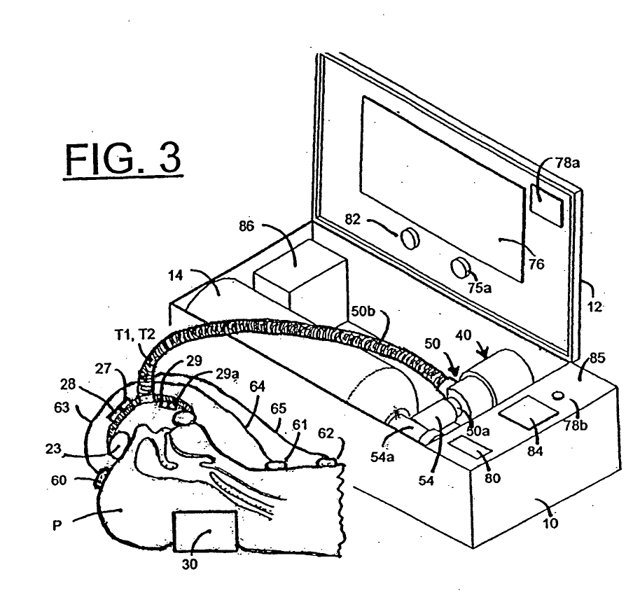

As indicated earlier, the emergency medical kit illustrated in the drawings is designed primarily (but not exclusively) for use by a non-professional person, such as a bystander, for rendering a respiratory and/or defibrillator treatment to a patient or other person under emergency conditions. The emergency medical kit, as shown in FIGS. 1 and 3, includes a housing, generally designated 10, having a cover 12 for opening the housing in order to provide access to its various components. When cover 12 is closed, it provides a relatively small, compact, portable unit that may be conveniently carried to the scene of an emergency, or that may be conveniently stored in a suitable nearby location for use during an emergency. The main operational components of the illustrated emergency medical kit are more particularly shown in the block diagram of FIG. 2.

As further shown in FIGS. 1 and 3, housing 10 includes a container 14 of pressurized oxygen. Container 14 is normally retained within housing 10 during use of the kit, but can of course be removable therefrom, e.g., for replacement or recharging purposes.

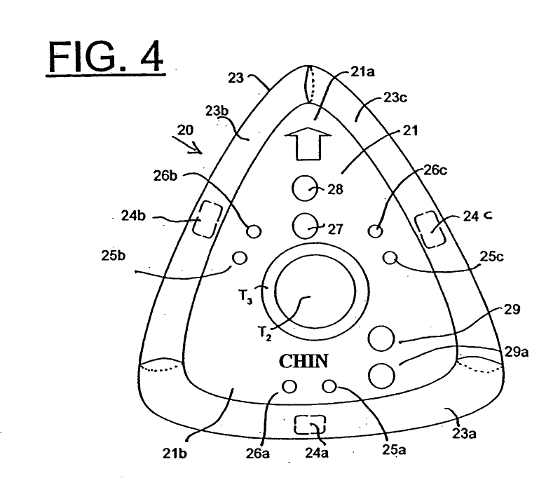

Housing 10 further includes a face mask, generally designated 20 and more particularly illustrated in FIG. 4. Face mask 20 is removable from housing 10 for application to the face of a patient requiring the emergency medical treatment, as shown in FIG. 3.

Face mask 20 is not viewable in FIG. 1 since it is covered by a neck rest, generally designated 30, which is also removably disposed within the housing and which must be removed before access is provided to the face mask underlying the neck rest. The neck rest is configured for supporting the neck of a patient in need of medical treatment, when the patient is in a reclining position, to facilitate application of the face mask to the patient, the delivery of oxygen for low-resistance inhalation by the patient, and the low-resistance discharge to the atmosphere of the exhalations of the patient.

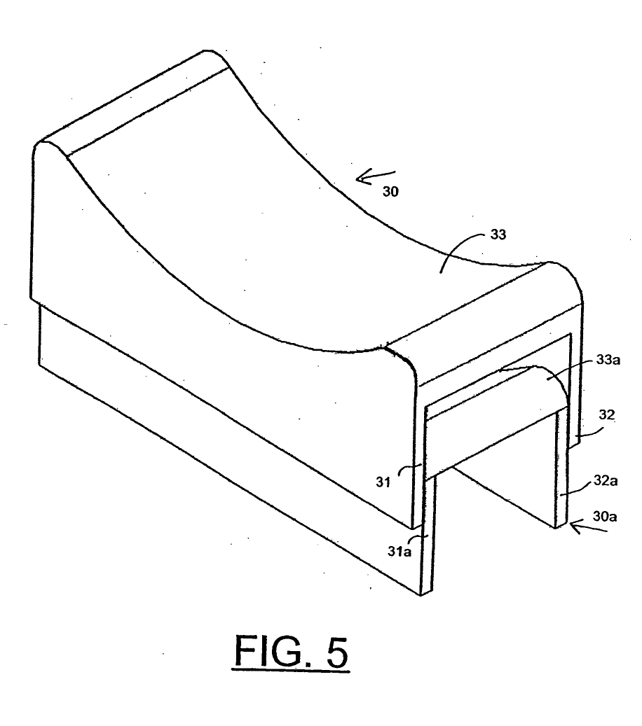

Preferably, the illustrated emergency medical kit includes two such neck rests as shown in FIG. 5 to be described more particularly below. One neck rest is dimensioned for use with an adult, and the other is dimensioned for use with a child.

The illustrated emergency medical kit further includes a respiratory pump, generally designated 40, within housing 10. Respiratory pump 40 is controlled by a valve assembly, generally designated 50, to connect, via a plurality of flexible feed tubes, the pressurized-oxygen container 14 to the face mask 20 such that the pump is driven by the pressurized oxygen to supply oxygen to the face mask for inhalation by the patient, and to discharge the exhalations of the patient via the face mask to the atmosphere. Pump 40 and valve assembly 50 controlling it are normally retained within housing 10 during the use of the emergency medical kit, but of course can be removable therefrom, e.g., for replacement or repair purposes.

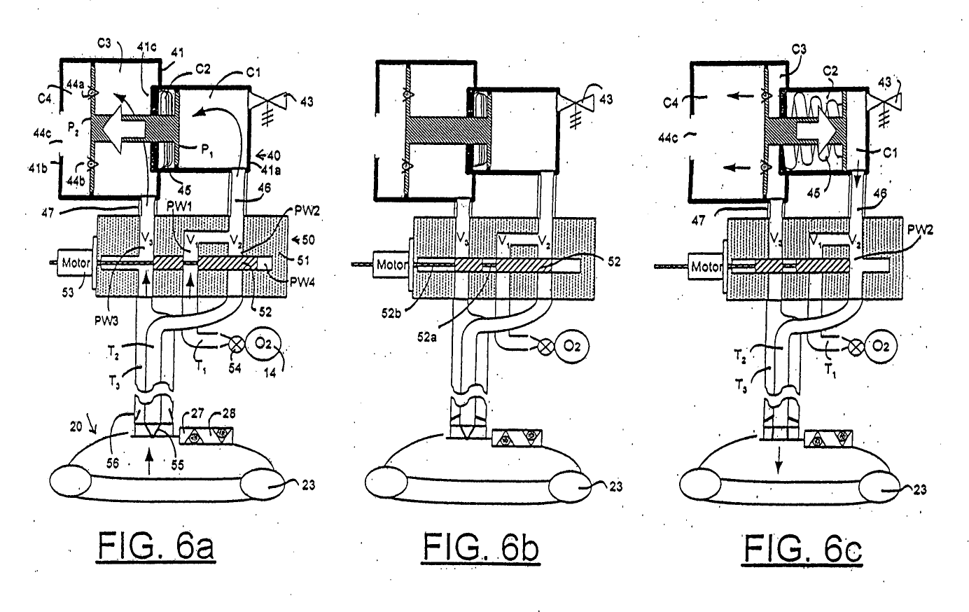

Thus, as shown particularly in FIG. 3, pump 40 is connected to the pressurized-oxygen container 14, via the valve assembly 50, by means of a first tube 50a (which corresponds to tube T₁ in FIGS. 6a-6c described below). Tube 50a remains within the housing 10, with the container and the pump, during the normal use of the kit when rendering an emergency treatment; whereas pump 40 is connected by a long flexible tube 50b (containing the two feed tubes T₂, T₃ described below with respect to FIGS. 6a-6c) to the face mask 20 to enable the face mask to be removed from the housing 10 and applied to the face of the patient P requiring the treatment.

The foregoing components of the illustrated emergency medical kit may be used for rendering respiratory assistance to a patient whenever required. The illustrated kit, however, is particularly useful for performing cardio-pulmonary resuscitation (CPR) and/or cardiac defibrillation under emergency conditions. For this purpose, the illustrated kit further includes a pulse-oximeter detector probe 60 for application to the patient (e.g., the patient's head as shown in FIG. 3) in order to detect the patient's pulse. The illustrated kit further includes a plurality of electrodes, designated 61 and 62 in FIG. 3, for administering electrical pulse therapy, e.g., defibrillation pulses, to the patient. Electrodes 61, 62 are also used for diagnosing and monitoring the electro-cardiac condition of the patient by detecting the patient's heart signals.

As shown in FIG. 2, the overall operation of the various components of the system included in the kit are controlled by a microprocessor, generally designated 70, also included within housing 10. Microprocessor 70 includes a number of inputs from the patient P, as illustrated by inputs 71 in FIG. 2, as follows: carbon dioxide (CO₂) concentration in the exhalations, as detected by a CO₂ detector 29a (FIG. 4) in the gas exhalation path of the face mask 20 as will be described more particularly below; pulse signals from the pulse-oximeter detector probe 60 (FIG. 3), which is preferably a pulse-oximetry detector; and heart signals which may be detected by the electrodes 61, 62.

Microprocessor 70 includes a further input signal 72 from the face mask 20 indicative of the seal pressure in each of three seal compartments of its inflatable seal, as will be described more particularly below. Microprocessor 70 also receives a ventilation pressure and flow signal 73, from a sensor 29 (FIG. 4) inside the face mask 20, as will also be described more particularly below.

The main output of microprocessor 70 is a ventilator control signal 74 which controls the valve assembly 50 to control the respiratory pump 40. Microprocessor 70, however, includes a number of additional outputs, as follows:

An output signal is produced to the defibrillator, as shown at 75, e.g., the cardiac electrode 61, 62 (FIG. 3), when a defibrillator activating button 75a (FIGS. 1, 3) is depressed.

An output is also produced to a visual display 76 for displaying operational instructions to the operator generated by microprocessor 70 during its operation. The outputs of microprocessor 70 are further monitored by a main BIT (built-in-test) module 77 which automatically monitors any fault in the system and controls, via the microprocessor, an audio instruction and/or alarm module 78. The main BIT module 77 is backed-up by an independent BIT module 77a which is responsible to produce an independent alarm if the main BIT module 77 fails. A failure is defined as existing when module 77 fails to send module 77a a signal during a predetermined time interval.

Microprocessor 70 further includes an output to a data logger module 79, which records all the patient records and treatments received by the patient during the event.

As further shown in FIG. 2, microprocessor 70 may also be used for enabling remote telephone communication and/or GPS location, via a remote telephone communication module 80 and GPS locator module 81. Thus, the emergency medical kit may be used for communicating with professional persons at a remote location, e.g., via a cellular telephone, for receiving treatment guidance, for advising such remotely-located persons of the exact location of the patient receiving the emergency treatment, and/or for directly defibrillating the patient.

As shown in FIGS. 1 and 3, the visual display 76 is located on the inner surface of the cover 12 so as to be viewable when the cover is opened. It may be a touch screen for inputting data. The inner face of the cover further includes an alarm indicator 78a, such as a flashing light, to produce an optical alarm, or a speaker to produce an audio alarm, one or both of which may be activated by the alarm module 78 (FIG. 2) upon the occurrence of an alarm condition.

The inner face of cover 12 further includes a manual control button 82 which may be depressed to start the system, to open an electric valve 54a (FIGS. 1, 3) between the oxygen container 14 and the pressure regulator 54, and to input, (e.g., prior to or during the medical treatment) basic information relating to the patient being treated and the medical scenario involved, e.g., cardiac arrest, respiratory emergency, etc. The microphone of the telephone communication 80 enables this data to be verbally inputted and recorded.

As the system should always be kept at a high reliability level, ready for operation in a very short notice, the apparatus is always in a self-testing condition by the main BIT 77, as well as by a back-up BIT 77a. Usually, cover 12 is closed during the standby condition of the kit. If a fault is diagnosed by the BIT module 77, the fault will be indicated on a screen 84, and/or by the light indicator 78b, carried by a ledge 85 of the housing 10 which is not covered by the cover 12 in the closed condition of the cover. In addition, if alarm 78a carried by the cover is an audio alarm, this alarm will also be actuated.

To enable the kit to be used during emergency conditions, the kit also includes its own battery power supply, shown at 86 in FIGS. 1-3. If the kit is to be carried by a vehicle, the kit could include merely a connector to the vehicle battery, e.g., via the cigar lighter terminal.

The Face Mask 20 (FIG. 4)

The construction of face mask 20 is best seen in FIG. 4. It includes a rigid transparent frame plate 21 of generally triangular configuration such that the narrow end 21a covers the patient's nose, and the wide end 21b is aligned with the patient's chin so as to cover the patient's mouth. Triangular frame 21 is formed with an opening for receiving the ends of two feed tubes T₂, T₃ to be received in the mouth of the patient.

Face mask 20 further includes a flexible inflatable seal 23 around the circumference of plate 21 engageable with the face of the patient receiving the mask for sealing the interior of the mask with respect to the outside atmosphere. Inflatable seal 23 is divided into three separate sections or air compartments 23a, 23b, 23c, each including a pressure sensor 24a, 24b, 24c, respectively. Each pressure sensor controls, via microprocessor 70, a green-light indicator 25a-25c or a red-light indicator 26a-26c according to the pressure sensed by the respective sensor. Thus, if the mask is properly applied over the patient's face, the pressure in all three compartments 23a-23c will be above a predetermined value needed for proper sealing, and therefore all three green indicator lights 25a-25c will be energized. On the other hand if any side of the mask is not properly pressed against the patient's face, the red indicator light 26a-26c for the respective compartment will be energized rather than the green indicator light.

Mask 20 illustrated in FIG. 4 further includes a maximum negative-pressure release valve 27, and a maximum positive-pressure release valve 28 to release the pressure within the mask should it exceed a predetermined negative or positive pressure. Mask 20 further includes a pressure sensor 29 for sensing the pressure within the mask, and a carbon dioxide (CO₂) sensor 29a for sensing the CO₂ concentration of the exhalations. The sensed pressure and CO₂ concentration are inputted into microprocessor 70 via input line 73 (FIG. 2).

As shown particularly in FIG. 3, face mask 20 also carries the pulse-oximeter detector probe 60 and the plurality of electrodes 61, 62 so as to facilitate their deployment when the face mask is removed from housing 10 for application to the face of a patient requiring the medical treatment. Thus, pulse-oximeter detector probe 60 is connected, via microprocessor 70, to the power supply 86 within housing 10 by an electrical conductor 63 carried by the flexible feed tube 50b connecting the mask to the pump 40, and electrodes 61, 62 are similarly connected by electrical conductors 64, 65 carried by the flexible feed tube 50b. Such an arrangement is not only compact for accommodation within housing 10, but also greatly facilitates the application of the probe 60 and electrodes 61, 62 to the patient when the mask 20 is removed from the housing for application to the patient.

The Neck Rest 30 (FIG. 5)

While FIGS. 1 and 3 illustrate only a single neck rest 30 included in the emergency medical kit, preferably there would be two (or more) such neck rests, as illustrated by neck rest 30 and 30a in FIG. 5. Both neck rests 30 and 30a are similarly configured for supporting the neck of a patient when in a reclining position. Neck rest 30 would be dimensioned for supporting the neck of an adult, whereas neck rest 30a would be dimensioned for supporting the neck of a child. As shown in FIG. 5, they are configured so as to be in a nested relationship when disposed within housing 10 of the emergency medical kit.

Thus, as shown in FIG. 5, each of the neck rests 30, 30a, includes a pair of spaced, parallel side walls 31, 32, 31a, 32a engageable at their lower ends with a horizontal surface, e.g., the ground or floor, receiving the patient in a reclining position. Both neck rests further include an upper wall 33, 33a, of concave configuration for supporting the neck of the patient when received in the reclining position, as shown in FIG. 3.

As indicated earlier, neck rest 30 (preferably with neck rest 30a) is disposed within housing 10 of the emergency medical kit to overlie the face mask 20, as shown in FIG. 1. This better assures that the neck rest will first be removed from the kit so as to enable it be to properly deployed to receive the patient, before the face mask is removed from the kit for application to the patient.

The Respiratory Pump 40 (FIGS. 2 and 6a-6c)

As indicated earlier, respiratory pump 40 included within housing 10 of the emergency medical kit is controlled by valve assembly 50 so as to be driven by the pressure within the pressurized-oxygen container 14, to supply oxygen from the pressurized-oxygen container 14 to the face mask 20 for inhalation by the patient, and to discharge the exhalations of the patient to the atmosphere. This is all done in a controlled manner as will be described more particularly below in the description of the overall operation of the system.

Respiratory pump 40 includes a pump housing 41 having an end wall 41a at one end, an end wall 41b at the opposite end, and a partition wall 41c between the two end walls. Pump 40 further includes a first piston P₁ movable between end wall 41a and partition wall 41c, to define a first chamber C₁ with end wall 41a and a second chamber C₂ with the partition wall 41c. Pump 40 further includes a second piston P₂ movable between partition wall 41c and end wall 41b, to define a third chamber C₃ with the partition wall 41c, and a fourth chamber C₄ with end wall 41b. Pump 40 further includes a stem 42 coupling the two pistons P₁, P₂ for reciprocation together.

Chamber C₁ includes a pressure-release valve 43 to prevent an excessive pressure within that chamber. Chamber C₂ is preferably continuously vented to the atmosphere.

Piston P₂ carries one or more one-way valves 44a, 44b, permitting air flow from chamber C₃ into chamber C₄, but blocking air flow from chamber C₄ to chamber C₃.

Respiratory pump 40 further includes a spring 45 interposed between piston P₁ and partition wall 41c for urging piston P₁ to contract chamber C₁ and expand chamber C₂. As will be described more particularly below, piston P₁ (and with it piston P₂) is driven in one direction by the high-pressure of the oxygen container 14, and is driven in the opposite direction by spring 45.

Respiratory pump 40 further includes a tube connector 46 leading into chamber C₁, and a second tube connector 47 leading into chamber C₃.

The Valve Assembly 50 (FIGS. 2 and 6a-6c)

Valve assembly 50, which controls respiratory pump 40, includes a block 51 formed with a plurality of passageways PW₁, PW₂ and PW₃, therethrough. Valve assembly 50 further includes a valve member 52 movable within a further passageway PW₄ in block 51 by means of an electrical motor 53 to control fluid flow through passageways PW₁-PW₃. Thus, valve member 52 is in the form of a cylindrical stem having reduced-diameter cylindrical sections 52a, 52b, to define three valves V₁, V₂, V₃ with respect to passageways PW₁, PW₂, PW₃, respectively, which may be selectively opened or closed, according to the position of valve stem 52 within passageway PW₄.

Passageway PW₁ is connected at one end to the pressurized-oxygen container 14 via a tube T₁ and a pressure regulator 54. The opposite end of passageway PW₁ is connected to passageway PW₂ at the side thereof facing the respiratory pump 40.

Passageway PW₂ is connected at the latter end to tube connector 46 of the respiratory pump 40, and at the opposite end to the face mask 20 via flexible tube T₂. Passageway PW₃ is connected at one end to tube connector 47 of the respiratory pump 40, and at the opposite end via a flexible tube T₃ to the face mask 20. Tube T₁ corresponds to tube 50a shown in FIGS. 1 and 3, whereas tubes T₂ and T₃ are disposed coaxially within the long flexible feed tube 50b shown in FIGS. 1 and 3. Flexible feed tube 50b also carries the conductors from sensors 29, 29a, 60, 61 and 62 to microprocessor 70.

As shown schematically in FIGS. 6a-6c, which will be described below in connection with the description of the overall operation of the system, the end of tube T₂ includes a one-way valve 55 which permits only inflow of gas (oxygen, as described below) into the face mask, and a second one-way valve 56 which permits only outflow of gas (exhalations) from the mask into tube T₃. Thus, a minimum dead space of inflow and outflow gases is achieved.

Valve stem 52 is reciprocated by electrical motor 53 under the control of the ventilator control signal 74 outputted from microprocessor 70. Thus, when motor 53 moves valve stem 52 to the extreme right position as illustrated in FIG. 6a, valves V₁ and V₃ are open, and valve V₂ is closed; whereas when the motor moves the valve stem to the extreme left position as illustrated in FIG. 6c, valves V₁ and V₃ are closed, whereas valve V₂ is open. The opening and closing of these valves drives the respiratory pump 40 to supply oxygen for inhalation by the patient, and to discharge the exhalations of the patient to the atmosphere, as will be described more particularly below.

The Overall Operation

As indicated earlier, the illustrated emergency medical kit continuously makes a self-check by means of the main BIT module 77, and the independent BIT module 77a. If a fault is found to be present by module 77, this information will be displayed on screen 84 and/or indicated by the light indicator 78b, even when the cover 12 is in its closed condition closing the housing 10. Independent BIT module 77a has an independent power source and alarm system, and continuously monitors the routine operation of module 77. In case module 77a detects a problem in module 77, module 77a activates its independent alarm system.

When an emergency condition occurs, cover 12 is opened. At that time, the user may input basic information relating to the patient needing the emergency treatment and the medical scenario existing (e.g., cardiac arrest, approximate age of the patient), etc. This information may be inputted via the touch screen 76 or the microphone at the remote telephone communication 80, may be recorded in the data logger module 79, and may be transmitted via the telephone communication unit 80 to a remote location. The specific location of the episode may also be communicated as determined by the GPS locator 81.

Neck rest 30, or both necks rests 30, 30a (FIG. 5), are removed and the appropriate one (adult or child) is placed under the neck of the patient while in a reclining position. The face mask 20, thus rendered accessible by removal of the neck rest 30, is then applied to the patient's face, with the end of the feed tube 50b (containing tubes T₂, T₃, FIGS. 6a-6c) received in the patient's mouth, and the peripheral seal 23 firmly pressed against the patient's face. If the seal is not properly applied so that one or more sides of the face mask are not firmly pressed against the patient's face, this will be indicated by the energization of a red indicator lamp 26a-26c at the respective side of the mask, rather than a green indicator lamp 25a-25c. Accordingly, the operator will be able to immediately discern and correct any improper sealing of the face mask with respect to the patient's face. In addition, an audible signal will be automatically transmitted by module 78a, and a visual demonstration will be displayed on screen 76.

The pulse-oximeter detector probe 60 is then applied, e.g., at the top of the patient's head as shown in FIG. 3, and the cardiac electrodes 62, 63 are also applied to the patient's chest.

If needed, the defibrillator module 75 (FIG. 2) may be activated by depressing defibrillator button 75a on the inner face of the cover 12 (FIGS. 1, 3). The operator may also communicate with a professional health care person to inform that person of the situation and to receive further instructions via the audio instruction and alarm module 78.

In addition, if no breathing is detected by the carbon dioxide sensor 29a, the respiratory pump 40 is activated by depressing button 82 to activate the valve assembly electric motor 53. Motor 53 reciprocates valve stem 52 of the valve assembly 50 first in one direction to one limit position (e.g., as shown in FIG. 6a) and then in the opposite direction to the opposite limit position (e.g., as shown in FIG. 6c). FIG. 6b merely illustrates an intermediate position between the two limit positions of FIGS. 6a and 6c. This reciprocation of valve stem 52 connects the respiratory pump 40 to the pressurized oxygen container 14, to utilize the energy therein to supply pressurized oxygen from container 14 into chamber C₁ for later inhalation by the patient, and to discharge exhalations of the patient from chamber C₃ to the atmosphere. This is done in the following manner:

Assuming valve stem 52 is in the limit position illustrated in FIG. 6a, in this position valves V₁ and V₃ are opened, whereas valve V₂ is closed. Thus, oxygen is supplied from the pressurized-oxygen container 14 via tube T₁ passageway PW₁ and connector 46 to chamber C₁ of the respiratory pump 40. This moves piston P₁ leftwardly, compressing spring 45, to expand chamber C₁ and to contract chamber C₂. Since piston P₁ is coupled by piston stem 42 to piston P₂, the latter piston will also move leftwardly, thereby expanding chamber C₃ and contracting chamber C₄. The expansion of chamber C₁ fills it with oxygen, and the expansion of chamber C₃ draws exhalations from the patient's mask 20 into chamber C₃ via tube T₃. The one-way valves 44a, 44b carried by piston P₂ are closed and thereby retain the exhalations within chamber C₃. Opening 44c in chamber C₄ permits a free contraction of chamber C₄, and also effects the discharge of the air (previous exhalations) within that chamber to the atmosphere during the next cycle.

Thus, during the stroke indicated by FIG. 6a, chamber C₁ is filled with pressurized oxygen, while chamber C₃ is filled with exhalations from the patient.

FIG. 6b illustrates an intermediate position of valve stem 52, wherein all three valves V₁, V₂ and V₃ in passageways PW₁, PW₂, PW₃ are closed.

FIG. 6c illustrates the opposite extreme position of the valve stem 52, wherein the previously open valves V₁, V₃ (in passageways PW₁, PW₃,) are now closed, and the previously closed valve V₂ (in passageway PW₂) is now open.

In this condition of the respiratory pump, the closing of passageway PW₁ disconnects chamber C₁ from the pressurized-oxygen container 14, thereby permitting the spring 45 within chamber C₂ to move piston P₁ rightwardly, as shown in FIG. 6c, to contract chamber C₁, and to force the oxygen therein via connector 46, passageway PW₂, and tube T₂ into the patient's mouthpiece for inhalation by the patient.

In addition, piston P₂, rigidly coupled to piston P₁, also moves rightwardly, thereby contracting chamber C₃ and expanding chamber C₄. This movement of piston P₂ permits the gas (exhalations) within chamber C₃ to be transferred via the one-way valves 44a, 44b into chamber C₄, for discharge from that chamber through opening 44c.

As noted earlier, chambers C₁ and C₂, as well as the piston P₁ movable therein, are of smaller cross-sectional area than chambers C₃, C₄ and piston P₂. Thus, the volume of chamber C₁ is less then that of chamber C₃ so that the pressure within chamber C₃ is less than that within chamber C₁. The control of the end pressure in chamber C₁ permits not only to regulate inhalation pressure, but also to regulate the exhalation pressure such that it can even be made sub-atmospheric to aid exhalations from the patient. This control can be effected by controlling the electric motor 53, via the control signal outputted by microprocessor 70 to the ventilator control module 74, to control the movements of the reciprocatory stem 52 of the valve assembly controller 50.

Such an action particularly when accompanied by chest compressions, increases the chances of patient survival if the emergency treatment is taken in time. As shown above, the respiratory pump may be operated according to an Active Compression-Decompression (ACD) mode, and also according to a Positive End-Expiratory Pressure (PEEP) mode, with relative low electric power consumption.

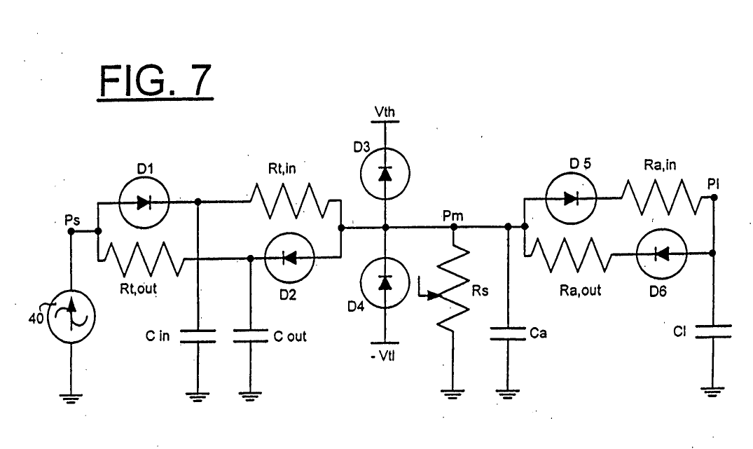

The Electronic Model (FIG. 7)

The electronic model of the pneumatic and of the respiratory systems is shown in FIG. 7, schematically indicating the pump 40 (having a sinusoidal operation), the circuit pressure elements, circuit compliance elements, and the one-way flow elements (diodes).

Pm is the pressure in the mask bulk (cmH2O); Pl is the lung pressure (cmH2O); Cin, Cout, C₁ and C_{b} are the entering circuit compliance through connector 46 and tube T₂ and the exhaust circuit compliance through connector 47 and tube T₃, lung compliance and patient's airway and mask compliance, respectively (liters/cm H2O); Rm is the mask sealing resistance (cmH2O/liter/sec) represents the rate of sealing; (when the mask is sealed completely, Rm can be considered as infinite); Ra,in and Ra,out are the patient's airway resistance during inhalation and exhalation phases, respectively (cmH2O/liter/sec); Rt,in and Rt,out are the connecting circuit resistances during inhalation and exhalation, respectively (cmH2O/liter/sec); and Ps is the pressure (sinusoidal) supplied by the ventilator's pump 40 (cmH2O). D1, D2, D3, D4, D5 and D6 are ideal diodes. D1 and D5 are conductive during inhalation, while D6 and D2 are in closed condition, and verse versa, during exhalation process. D3 is opened when the pressure in the mask exceeds a maximum positive threshold pressure of Vth, as determined by value 42. D4 is opened when the pressure inside the mask is below a maximum negative threshold pressure—Vth, as determined by valve 43.

The model derives a real time estimate of patient airway resistance, lung compliance, connecting tube compliance and lung elasticity. These estimates are used to regulate pump parameters in real time using closed loop monitoring of output signals.

The illustrated apparatus is able to provide consistent regulation for each individual patient throughout the whole medical treatment as well as automatically adjust itself for wide range of different patients, from small infants to large adults.

Sinusoidal pump 40 is able to provide positive and negative end-expiratory pressures by selecting the oxygen chamber end pressure C₁, taking into consideration the system fixed parameters (the increased volume for the same displacements of pistons P₁ and P2, as well as the dynamic parameters at each instant of operation.

The illustrated model allows different-resistances at inhalation and exhalation phases. Separation of inhalation and exhalation resistances results from different diameter of the inhalation and exhalation tubes and from possible elasticity differences of the lungs and thorax during inhalation. Prior art considers inhalation and exhalation airway resistances in two alternatives: as linear or as non-linear functions. Recognizing the fact that gas flow through an endotracheal tube may be turbulent, a non-linear approach is recommended:
P(F)=Kₚ*f(t)ⁿ
P(F) is the pressure due to the flow across the patient airway, f(t) is the bi-directional flow, n is an empirically invariant exponential (usually ranged between 1.4 to 1.7) and Kp is constant within a single breath.

When analyzing the above electronic circuit by using well-known Kirchoff's voltage laws and transferring the results to the frequency domain, a transfer function of circuit pressure to flow at the mask area can be found for inhalation and exhalation phases.

Pressure and flow rate sensors signals may be transmitted via an analog to digital converter (AD converter) and an anti-aliasing filter. These inputs enable the microprocessor to calculate Cin, Cout, Ca, Ra,in, Ra,out and the mask leakage volume—using standard analytical techniques, such as the recursive least squares method. The output signal generates by the microprocessor are used for controlling and optimizing the pump parameters. Closed-loop method is used for minimizing the error signal and for achieving desired parameters at real time.

It will be appreciated that the described apparatus is very user-friendly, and does not require a high degree of skill or experience on the part of the user. The system may be provided with other detectors and sensors to detect other medical conditions, such as airway obstructions indicated by high impedance pressures, carbon dioxide detectors 29a to analyze the exhalations and thereby to indicate the return of a spontaneous pulse and to prevent hyperventilation. In addition, heart signal sensors may be provided to monitor the cardiac electrical signals.

Pulse-oximeter probe 60 may be an optical oximetry type detector to monitor the oxygen level of the blood, which would thereby also facilitate the early detection of the return of natural circulation on the part of the patient.

All the operations of the described apparatus are controlled by microprocessor 70 which may be programmed to produce optimal ventilation pressures and flows for patients of different ventilation requirements and involved in different medical scenarios of cardiac arrest and respiratory emergencies.

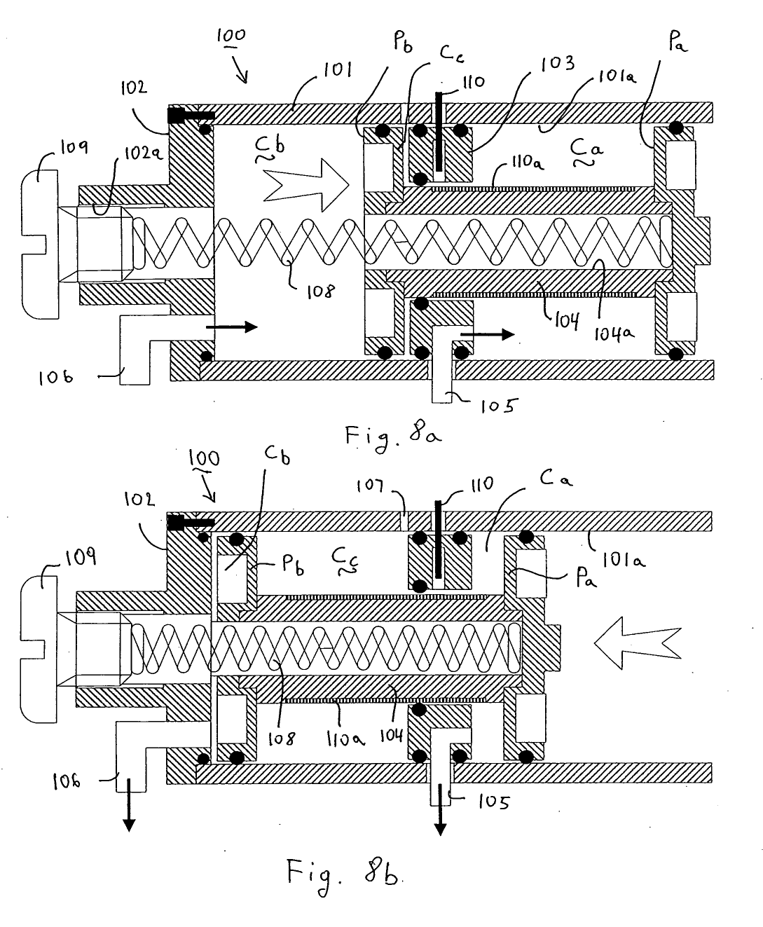

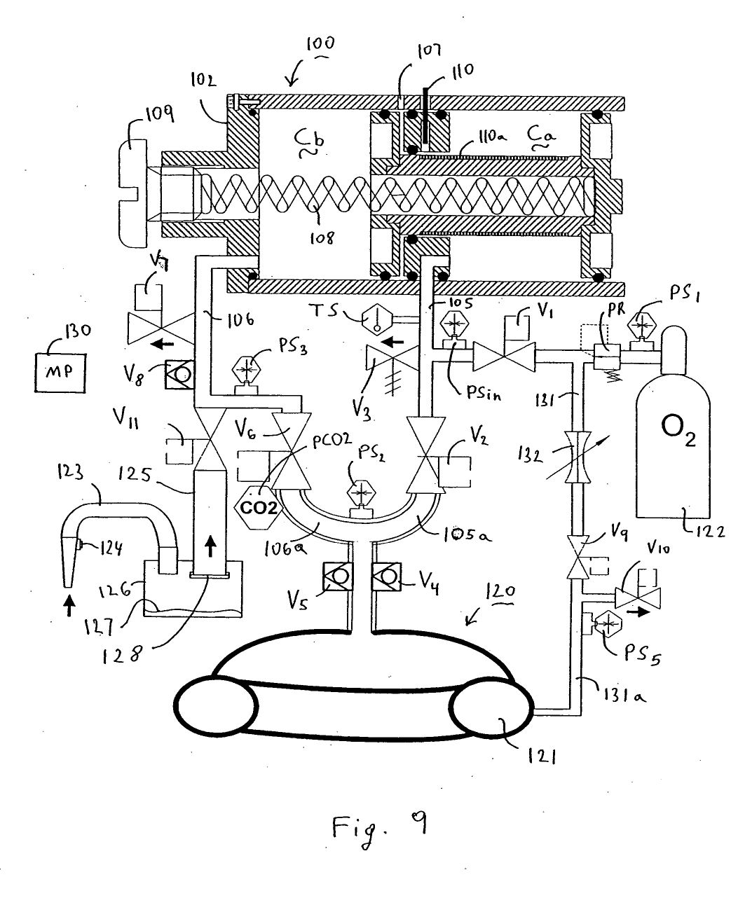

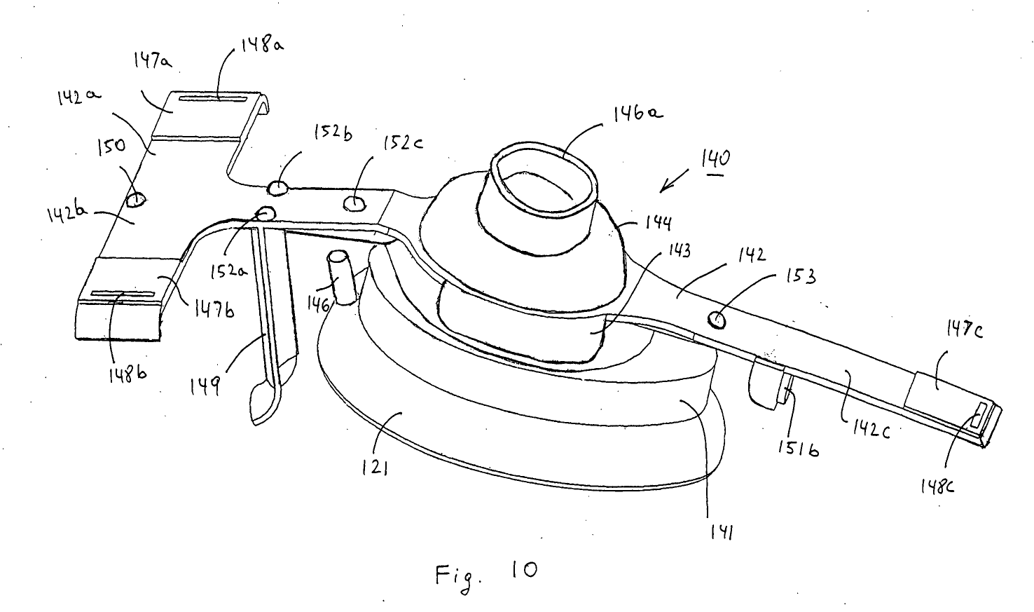

### The Embodiment of FIGS. 8a, 8b and 9

FIGS. 8a and 8b illustrate the end stroke positions of another respiratory pump, generally designated 100, which may be used in the emergency medical kit, instead of the respiratory pump of FIGS. 6a-6c. In principle both respiratory pumps are similar but: (a) the inner dimensions are the same for both chambers Ca and Cb in FIGS. 8a and 8b; (b) the inner spring (108) allows the deletion of chamber C2, of FIGS. 6a-6c; and (c) the motor-driven valve assembly of FIGS. 6a-6b is replaced by separate electric valves (e.g. V1, V2, V6) in FIG. 9. The advantages of the latter embodiment include: easier to manufacture, more compact, and more efficiently controlled for pressure adjustment.

FIG. 9 illustrates the main components of a system including the respiratory pump of FIGS. 8a, 8b. FIG. 8a illustrates the condition of the respiratory pump 100 during its exhalation phase, whereas FIG. 8b illustrates its condition during the inhalation phase.

Respiratory pump 100 illustrated in FIG. 8a and 8b includes a cylinder 101 open at one end, closed at the opposite end by an end wall 102, and formed with an intermediate partition 103. The illustrated respiratory pump further includes two pistons Pa, Pb coupled together by a stem 104 passing through partition 103, such that piston Pa defines a first chamber Ca with partition 103, and piston Pb defines a second chamber Cb with the end wall 102. The net volume of chamber Ca equals the inner volume of cylinder 101a with piston Pa minus the corresponding stem 104 volume included in chamber Ca. Thus, low-pressure chamber Cb volume is always greater than that of the high pressure of chamber Ca. The design of the volume ratio Cb/Ca is done by selecting the diameter of 101a and the diameter of stem 104. When no leakage exists during ventilation, inhalation gas mass at Ca equals the exhalation gas mass at Cb.

Piston Pb also defines a third chamber Cc between it and partition 103. As will be described more particularly below with respect to FIG. 9, chamber Ca is adapted to receive a supply of oxygen via a port 105 for use during the inhalation phase, whereas chamber Cb is adapted to receive the exhalation gas from the patient via a port 106 during the exhalation phase. Chamber Cc, between partition 103 and piston Pb, is vented to the atmosphere via an opening 107.

The illustrated respiratory pump further includes a spring 108 received within a passageway 104a and passing through chamber Cb as well as through an opening in end wall 102. One end of spring 108 is secured to piston Pa, whereas the opposite end is secured to a threaded fastener 109 threadedly received within opening 102a of end wall 102.

FIG. 8b illustrates that spring 108 urges piston Pa towards partition 103 so as to decrease the volume of chamber Ca, and urges piston Pb towards end wall 102 so as also to decrease the volume of chamber Cb, but to increase the volume of chamber Cc. The piston assembly, including the two pistons Pa and Pb with their coupling stem 104, are movable (rightwardly) to the position illustrated in FIG. 8a by the pressurized oxygen supplied via port 105 to chamber Ca, to thereby increase the volume of the two chambers Ca and Cb, while decreasing the volume of chamber Cc.

The end pressure within chamber Ca may be preset by rotating screw 109, to thereby change the force applied by spring 108 to piston Pa. The instantaneous position of the piston assembly (and thus the volumes of chambers Ca and Cb) is indicated by sensor 110 carried by partition 103 and sensible markings 110a carried on the outer surface of stem 104. Sensor 110 may be an optical sensor, whereupon sensible markings 110a would be optically-sensible markings, such as opaque rings; alternatively, sensor 110 could be a magnetic sensor, whereupon sensible markings 110a would be in the form of magnetic rings carried on the outer surface of stem 104.

FIG. 9 illustrates a system wherein the respiratory pump 100 is coupled to a face mask, generally designated 120, for controlling inhalations and exhalations of the patient to whom the face mask is applied. Face mask 120 illustrated in FIG. 9 also includes a flexible inflatable sealing ring 121 engageable with the face of the patient for sealing the interior of the mask with respect to the outside atmosphere. Although FIG. 9 illustrates the inflatable seal 121 as constituted of a single air chamber, it will be appreciated that the face mask may include the plural-section seal described above with respect to FIG. 4.

The system illustrated in FIG. 9 further includes a source of pressurized oxygen 122, used not only for supplying oxygen to the patient, but also for driving the respiratory pump 100 and for inflating seal 121. Thus, the pressurized oxygen supply 122 is coupled to the oxygen port 105 leading into chamber Ca of respiratory pump 100 and provides the energy for activation of the respiratory pump 100. Thus, the electric consumption of this device is low.

In the system of FIG. 9, the exhalation port 106 coupled to chamber Cb of the respiratory pump 100 is used not only for aiding the exhalations of the patient wearing the face mask 120, but also for drawing out fluids, such as saliva, blood or emesis, from the patient's mouth. For this purpose, the system illustrated in FIG. 9 further includes a suction tube 123 insertable into the mouth of the patient, while the face mask 120 is removed, and connectable to a sump 126 for drawing out fluids from the patient's mouth. Suction tube 123 carries an electric button 124 which may be manually-depressed, as will be described more particularly below, to start the suction operation. Conduit 125 leads into the upper end of sump 126 into which the withdrawn fluids are received, as shown at 127. The withdrawn fluids may be removed from sump 126 in any convenient manner. The end of conduit 125 is covered by a filter 128 to prevent any such fluids from being drawn into chamber Cb.

The operation of the system illustrated in FIG. 9 is controlled by a plurality of mechanical valves (V3, V4, V5, V8) opened or closed at predetermined pressures and various electrically-actuated valves (V1, V2, V6, V7, V9, V10, V11) under the control of a microprocessor schematically shown at 130 in FIG. 9.

Thus, the supply of oxygen from the pressurized tank 122 is controlled by electric valve V₁ which, at the proper time, connects the pressurized oxygen tank to port 105 of chamber Ca of respiratory pump 100. Electric valve V₂ connects port 105 to the inlet tube 105a of the face mask 120 for inhalation by the wearer of the mask. The gas circuit between the pressurized oxygen container 122 and port 105 of chamber Ca includes, not only valve V₁ but also a pressure sensor PS₁ which continuously senses the pressure within container 122, a pressure regulator PR which regulates the pressure of oxygen supplied to chamber Ca; a pressure sensor PSin which senses the pressure at chamber Ca, and a temperature sensor TS which senses the temperature of the gas flowing into chamber Ca Thus, pressure sensor PS, continuously monitors the suction pressure in container 122 to indicate a possible leakage condition; pressure regulator PR reduces the high pressure (e.g., 200 Bars) within container 122 to less than two Bars before inputted into chamber Ca; microprocessor 130 uses the data from pressure sensor PSin, sensor 110 (indicating the volume of chambers Ca), and temperature sensor TS to calculate the oxygen flux into chamber Ca;

If the input pressure into chamber Ca exceeds a predetermined upper limit, mechanical valve V₃ is automatically opened to thereby decrease the pressure accordingly.

After chamber Ca has been filled with oxygen, valve V₁ is closed and valve V₂ is opened to enable gas flow between chamber Ca and the inhalation tube 105a into the face mask 120.

The pressure of the oxygen supplied via inhalation tube 105a to face mask 120 is monitored by pressure sensor PS_{2.} Valves V₄ and V₅ ensure the pressure at the patient's airway opening stays within the maximum and minimum range selected. This range of pressures ensures that no lung damage (i.e., barotraumas) occurs. For example, the pressure of the oxygen within the face mask 120 should be held within the upper limit of +50 cm H₂O to −5 cm H₂O. If the high limit is exceeded, valve V₄ is opened; and if the pressure drops below the lower limit, valve V₅ is opened.

Exhalation tube 106a from the face mask 120 also includes a sensor of partial pressure of CO₂ (i.e., capnograph) in the exhaled air described as PCO2. Sensor PCO2 also can be used as one of the means to detect the return of spontaneous breathing by the patient. Exhalation tube 106a also includes an electric valve V₆ which, when opened, enables gas flow between face mask 120 and pump chamber Cb via port 106. Port 106 further includes an exhaust electric valve V₇ for exhausting the exhalations from the patient received within pump chamber Cb, when piston Pb moves towards wall 102. Port 106 further includes a security mechanical valve V₈ which prevents excessive negative pressure within pump chamber Cb, and a pressure sensor PS₃ which monitors the pressure within pump chamber Cb.

As indicated earlier, the face mask 120 is sealed to the patient's face by the inflatable seal 121. Seal 121 is supplied with oxygen from container 122 via a line 131 including a restrictor 132 and an electric valve V9 which is opened to enable gas flow between oxygen container 122 and the mask inflatable seal 121. The pressure within inflatable seal 121 is monitored by pressure sensor PS₅ within line 131a. The pressure line 131a further includes an electrically-operated valve V₁₀ which may be opened to lower the pressure within seal 121, or to exhaust the oxygen from the inflatable seal 121.

Microprocessor 130 allows real-time adjustment of the pressure of inflatable sealing ring 121 to be above the ventilation pressure by a predetermined value (e.g. 5 cm water) in order to enhance patient comfort and to reduce skin damage. This is done by continuously controlling valves V₉ and V₁₀ in order to enhance patient comfort and to reduce skin damage

The illustrated system includes a further electrical-control valve V₁₁ which starts “suction mode” operating by microprocessor 130, e.g., by manually depressing button 124 on suction tube 123, whenever the suction tube is to be used for drawing out fluids from the patient's mouth. Thus, depressing button 124 initiates a “suction mode” of operation of the system, wherein suction tube 123 is connected to the suction chamber Cb of respiratory pump 100 via its port 106, valve V₁₁, tube 125 and sump 126. This “suction mode” continues so long as button 124 is depressed. It will be appreciated that during the “suction mode”, when valve V₁₁ is open, valve V₆ would be closed to interrupt communication between the negative-pressure of pump chamber Cb and the face mask 120.

The fluids (e.g., gastric substances) drawn out from the patient's mouth via suction tube 123 are accumulated within sump tube 126, as shown at 127. Such substances may be removed from sump tube 126 in any suitable manner. Such substances are prevented from being drawn into the negative-pressure suction chamber Cb by filter 128 applied across the end of tube 125 communicating with chamber Cb.

The system illustrated in FIG. 9, including respiratory pump 100, oxygen container 122, and microprocessor 130, may be provided in an emergency medical kit as described above with respect to FIGS. 1-7, including the neck rest, housing, and other controls carried by the housing as described above with respect to FIGS. 1-7. Such an emergency medical kit may be used in the following manner for rendering emergency medical treatment to a patient.

When the illustrated system is to be used to aid in the respiration of the patient, face mask 120 is applied to the patient's face, and seal 121 is inflated by opening valve V₉ to establish communication with the oxygen within container 122. The pressure of the oxygen supplied to seal 121 is reduced by restrictor 132 and is monitored by pressure sensor PS₅. For example, seal 121 may be inflated to a pressure of about 1.05-1.4 atmospheres; when this pressure is attained, valve V₉ is closed. The pressure within seal 121 is monitored by pressure sensor PS₅ to open valve V₉ in order to maintain the desired pressure, or to open valve V₁₀ should the pressure within seal 121 be larger than that desired.

With face mask 120 properly applied, respiratory pump 100 may then be operated according to the respiration mode by the pressurized oxygen within container 122.

During the respiration mode of operation, microprocessor 130 controls the electric valves to drive respiratory pump 100 through the two strokes illustrated in FIGS. 8a and 8b, respectively. FIG. 8a illustrates the end of the stroke wherein the two chambers Ca and Cb are in their expanded condition as driven by the pressurized oxygen within container 122; whereas FIG. 8b illustrates the end of the stroke wherein the two chambers Ca, Cb are in their contracted condition as driven by the return spring 108. During the stroke in which the respiratory pump is driven towards its expanded condition as illustrated in FIG. 8a, chamber Ca is filled with pressurized oxygen from container 122, whereas chamber Cb produces a negative pressure applied to the patient to produce an exhalation from the patient into chamber Cb. During the stroke in which the pump is driven towards its contracted condition, as illustrated in FIG. 8b, the oxygen within chamber Ca is forced into the patient's face mask, thereby producing inhalation by the patient, whereas the exhalation from the patient in chamber Cb is discharged to the atmosphere by electric valve V₇.

It will be seen that by appropriately controlling the timing of the electric valves, as well as the force applied by spring 108, the illustrated system can be controlled to produce various types of operations, including: (a) a positive end expiratory pressure (PEEP) operation; (b) a negative end expiratory pressure (NEEP) operation; and (c) a standard ventilation operation. For example, high-end pressures at Ca and an early closing of valve V₆ (while opening valve V₇ to prevent negative pressure at chamber Cb) during exhalation, leads to a PEEP operation; whereas low-end pressure at Ca and late closing of valve V₆ leads to a NEEP operation.

The end pressure of the oxygen within pump chamber Ca may also be varied by adjusting screw 109 to change the force applied by spring 108 to the piston assembly. Sensor 110, which as indicated above could be an optical or magnetic sensor cooperable with the sensible markings 110a on stem 104 of the piston assembly, monitors the position of piston Pa and Pb with respect to the partition. Thus, using real-time data of pressure sensor PSin (of chamber Ca), pressure sensor PS3 (of chamber Cb), together with the positions of pistons Pa and Pb (by sensor 107), enables mass flow calculations to be made by microprocessor 130 with respect to both chambers Ca and Cb. If the calculated mass flow at Cb is less than the mass flow at Ca, leakage (probably between the face mask and the patient's face) can be assumed. In this case, microprocessor 130 increases the pressure at chamber Ca to increase mass flow of Ca and thus to compensate for that leak. Thus, even if a leakage exists, the patient continues to receive the same predetermined amount of oxygen.

Due to the capability to produce Negative End Respiratory Pressures (NEEP) by respiratory pump device 100, the system is capable of enhancing or producing some degree of cardiac blood flow, instead, or in addition to the blood flow derived by manual chest-compressions. Cardiac blood flow is caused by positive and negative pressures oscillations in the airway opening. Since the lungs surround the heart, expansion and deflation of the lungs changes the pressure around the heart, thereby producing blood flow into and out,of the heart. Such use of the described apparatus thereby produces a form of internal heart massage.

Thus, this use of the described apparatus generates “systole” (the heart is squeezed by high pressure ventilation in the lungs during inhalation) and also induced “diastole” (the heart is expanded) by low pressure (NEEP) in the lungs during exhalation.

“Assistance chest compression” is defined as the above activations at moderate pressure conditions while being supported with conventional manual chest compression. When ventilation is performed at more extreme pressure conditions, a complete “Automatic chest compression” is achieved. These extreme high and low pressures should, of course, not be such as to risk damage to the lungs or to cause gastric inflation. “Assistance chest compression” and “Automatic chest compression” are preferably performed by producing, continuously, (a) 15 cycles of short high-pressure inhalation and negative expiratory pressure, followed by (b) 2 cycles of standard-pressure ventilation, as long as needed.

In the “Automatic chest compression” process, the single bystander only needs to connect the mask, the defibrillator and the pulse-oximeter; all the other essential bystander “chain of survival” operations are done automatically.

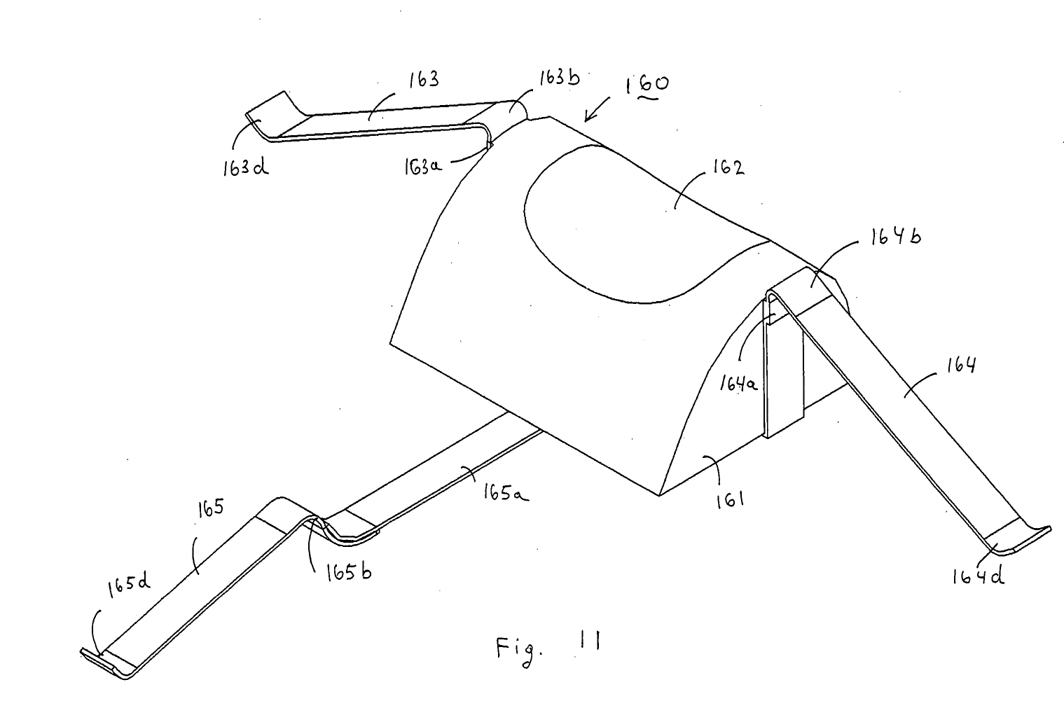

Modified Kit Constructions (FIGS. 10-19)

FIGS. 10-13 illustrates modifications in the structure that may be made in both the face mask and also in the neck rest. Such modified structures can be used in the emergency medical kit described above, or can be provided as stand-alone equipment for use with conventional ventilators, both in hospital and ex-hospital.

FIG. 10 illustrates an assembly, generally designated 140, which includes a face mask seal 121 and a mounting bracket 142 secured to the face mask, e.g., by a pair of securing members 143 and 144. Face mask 140 may be of any of the constructions described above, or of any standard face mask construction, but includes a flexible inflatable seal 121 having an inflation/deflation tube 146. Mask assembly 140 further includes an inhalation/exhalation tube 146a, which can also serve as a handle for manipulating the assembly.

Bracket 142 is of a three legs-configuration. It includes two legs 142a, 142b at one end in alignment with each other to project laterally from opposite sides of the mask assembly at that end, and a third leg 142c projecting from the opposite end of the assembly perpendicularly to the two legs 142a, 142b. Each of the legs 142a-142c includes a connector strip 147a-147c, respectively, such as of Velcro or the like, for connection to straps carried by the neck rest, as will be described more particularly below. Each of the legs 142a-142c may include a magnetic sensor 148a-148c cooperable with magnetic elements on the straps 163d, 164d, 165d of the neck rest 160 in order to effect and monitor a proper attachment of each strap to connector strip 147a-147c.

As will also be described below, mask assembly 140 is applied such that the two legs 142a, 142b are located at the lower end of the patient's face, whereas leg 142c is located to extend over the forehead of the patient's face. To assure proper orientation of the face mask assembly when applied to the patient, bracket 142 is provided with a chin alignment bar 149, extending perpendicularly from the bracket, to engage the undersurface of the patient's chin.

Bracket 142 may also carry a number of indicators, including those provided in the face mask illustrated in FIG. 4. Indicator 150 could be provided and controlled to flicker when the user is asked to install the defibrillator electrodes, shown at 150a, 150b in FIG. 12; indicator 153 could be provided and controlled to flicker when the pulse-oximeter sensor is to be applied to the patient's forehead, as shown at 151b in FIG. 12; and indicators 152a, 152b and 152c could be provided to display “green” or “red” as described in the following to indicate the proper or improper applications of the face mask.

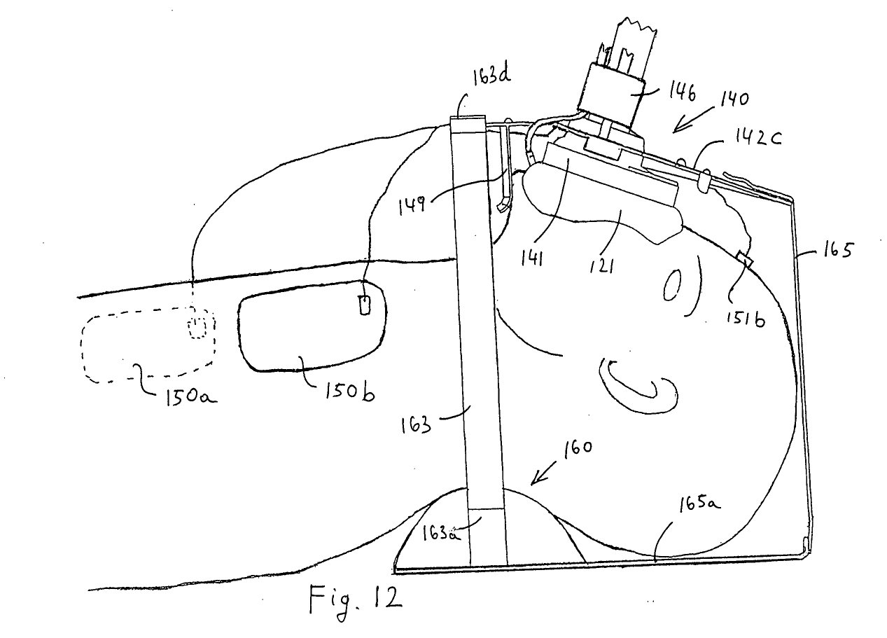

FIG. 11 illustrates the neck rest, generally designated 160, to be used with the face mask assembly 140 of FIG. 10. As described above particularly with respect to FIGS. 1-7, neck rest 160 in FIG. 11 is also configured for supporting the neck of a patient in need of medical treatment, when the patient is in a reclining position, to facilitate application of the face mask to the patient, the free delivery of oxygen for inhalation by the patient, and the free discharge to the atmosphere of the exhalations of the patient with minimum flow resistance.

Neck rest 160 in FIG. 11 may be of the relatively solid construction as described above with respect to FIG. 5, formed with a flat base 161 for stably resting on a flat horizontal surface, and a curved recess 162 at its upper end for receiving the neck of the patient. Alternatively, neck rest 160 could be of an inflatable construction, as described below particularly with respect to FIG. 14, so as to permit its deflation for compact storage and handling, and inflation at the site of use.

In either case, neck rest 160 is provided with three inelastic straps 163, 164, 165, arranged so as to be cooperable with the connector strips on the three arms 142a-142c, respectively, of the face mask assembly 140 (FIG. 10). Each inelastic strap 163-165 includes a fixed section 163a-165a connected to an inelastic, flexible section 163b-165b. The outer end of each of the three inelastic straps 163, 164, 165 is carried a magnetic connector strip 163d-165d cooperable with connector strip 147a-147c of the face mask assembly 140. For example, the cooperable strips on the neck rest 160 and face mask assembly 140 could be of “Velcro”

FIG. 12 illustrates the manner of deploying the neck rest 160 and the face mask assembly 140 when rendering emergency medical treatment to a patient. Thus, the neck rest 160 is applied under the patient's neck while the patient is in a reclining position. If desired, a plastic airway cannula (not shown) may be placed in the patient's mouth in order to secure airway passage to the lungs.

The face mask assembly 140 is then applied over the patient's face as shown in FIG. 12, with the chin rest 149 engaging the undersurface of the patient's chin, so that arm 147c of the face mask assembly overlies the patient's forehead. The three inelastic straps 163-165 of the neck rest are then firmly secured to the connector strips 147a-147c carried by the bracket arms 142a-142c of the face mask assembly 140. The three straps can be connected to the face mask assembly 140 in any order.

During the connection of the straps, the face mask assembly is lightly pressed against the patient's face to assure firm contact of seal 121 of the face mask assembly with the patient's face. The pressing of the mask assembly 140 over the patient's face causes a pressure rise within seal 121. This pressure rise is indicated by pressure sensor PS5 (FIG. 9) and is monitored by microprocessor 130. The engagement by each magnetic sensor 148a-148c with the magnetic straps 163d, 164d, 165d (of inelastic straps 163-165) is also monitored by microprocessor 130. By lightly pressing the mask during its application to the patient's face, a minimum volume increase is produced in the inflatable sealing ring 121 when it is inflated, thereby producing a good seal.

The proper attachment of each strap will be indicated by green lights 152a-152c; that is, if during the connection of each of the straps, the pressure in sealing-ring 121 is above-predetermined pressure, its light will turn green. On the other hand, if the mask was not properly pressed, a red light related to the last strap connected will be indicated. For example when strap 165 is not properly connected, indicator 152c will be red. This red indication will appear together with comments from the audio instruction 78 (FIG. 2) and on touch screen 76 (FIG. 1).

Shortly after the three straps are properly attached, sealing ring 121 is expanded to a predetermined pressure by valve V9. The pressure within the sealing ring 121 should be above the peak pressure of the oxygen applied to the face mask assembly during the inhalation phase, to prevent leakage during inhalation.

At this time, the defibrillator electrodes 150a, 150b, and the pulse-oximeter detector 151b, may be applied if desired. The proper application of the defibrillator electrodes and of the pulse-oximeter detector is indicated by indicators 150 and 153, respectively, of the face mask assembly 140, together with visual and audio instructions by 76 and 78 of the control system of FIG. 2.

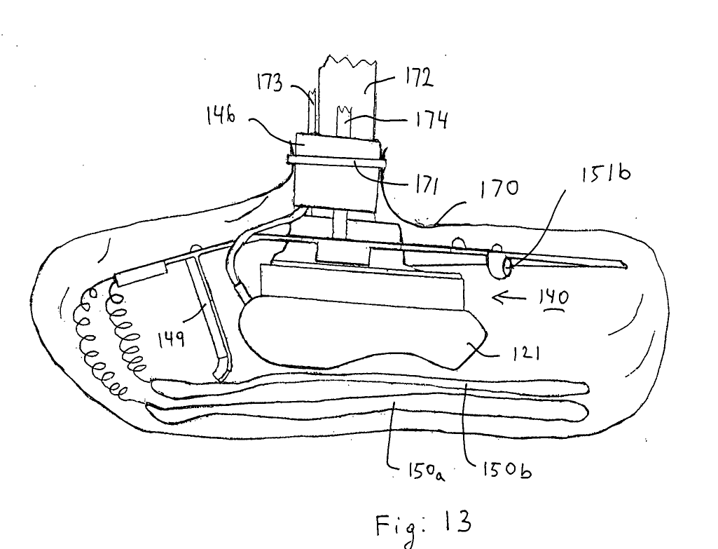

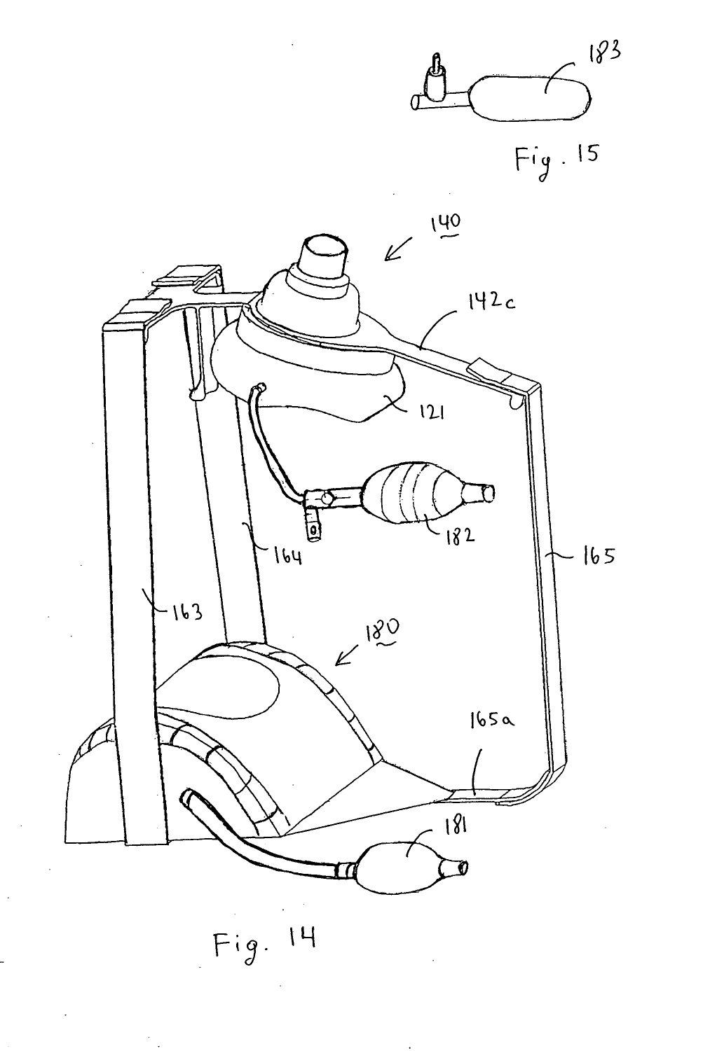

FIG. 13 illustrates one manner of packaging the face mask assembly 140 for long storage and handling, ready for use whenever desired. Thus, the face mask assembly 140 is stored within a plastic bag 170 which is hermetically sealed by a sealing band 171 applied around the handle 146 of the face mask assembly. As shown in FIG. 13, the inhalation/exhalation tubes 172 and 173, as well as the electrical conductors 174 leading to the various electrical components carried by the face mask assembly 140, are passed through handle 146. In order to improve storage conditions, valves V2 and V6 (FIG. 9) would be closed to prevent the entry of air into the bag.

Preferably, the defibrillator electrodes 150a, 150b, as well as the pulse-oximeter detector 151b, are electrically connected to the face mask assembly 140, so that they would also be contained within the medically-sealed plastic bag 170, ready for use whenever needed.

As indicated earlier, neck rest 160 used with face mask assembly 140 could be of a solid construction, as described above with respect to FIGS. 1-7, or of an inflatable construction. FIG. 14 illustrates the latter option, wherein it will be seen that the neck rest, therein designated 180, is of an inflatable construction. In this example, it is inflatable by manual pump 181.

FIG. 14 also illustrates the variation wherein the seal 121 of the face mask assembly 140 is also inflatable by a manual pump 182.

Instead of using a manual pump for inflating the neck rest 180 or the seal 121 of the face mask assembly 140, other inflation means could be used. FIG. 15 illustrates the variation wherein a gas-discharge cartridge 183 is used for inflating the neck rest and/or the seal of the face mask assembly.

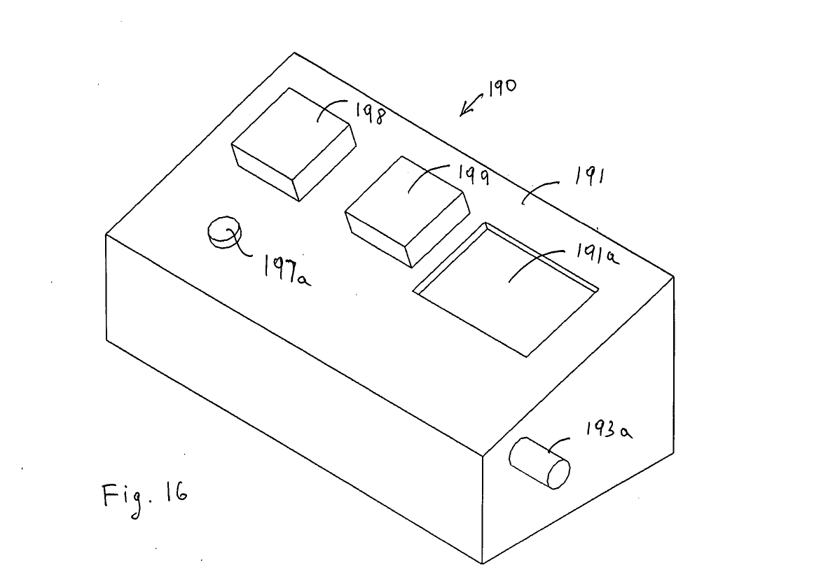

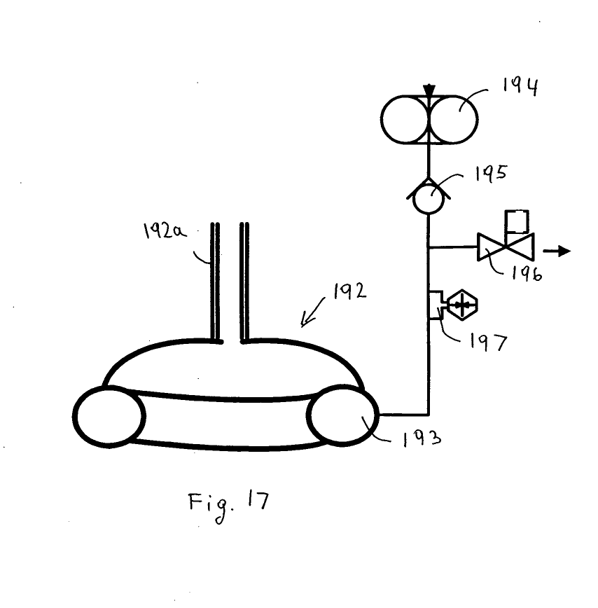

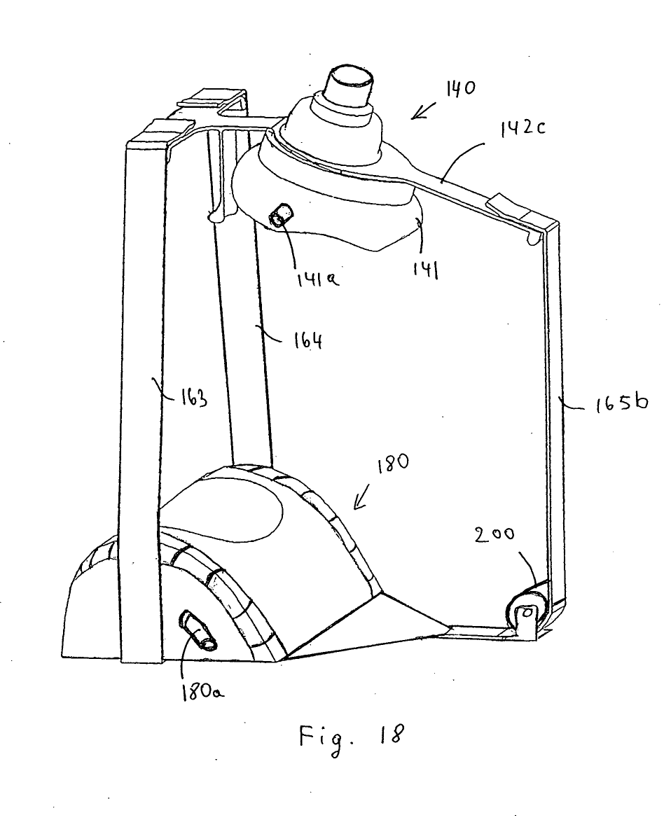

FIGS. 16 and 17 illustrate the components of a simplified, mobile, kit which is intended for automatic mask attachment described above. In this case, any kind of ventilator can be connected to the mask Mouth-to-mask ventilation is also possible. Such a kit, generally designated 190 in FIG. 16, includes a housing 191 for containing the face mask 192 (FIG. 17) having an inflatable seal 193, as described above. In this case, however, seal 193 is inflatable by a compressor 194 via a one-way valve 195. The air within seal 193 is exhausted by an electrically controlled discharge valve 196, and the pressure within the seal is indicated by pressure sensor 197.

As shown in FIG. 16, housing 191 includes a screen 191a displaying the pressure in seal 193 and also indicating faults, (e.g., low battery voltage). Also carried by housing 191 is an indicator 197a indicating that the pressure sensed by pressure sensor 197 is above a predetermined minimum pressure rise while the mask is pressed against the face. On/off button 198, and compressor actuator button 199 are also carried by housing 191. If the mask pressure is not sufficient, an light indication will be shown on indicator 191a and the activation of compressor 194 will be delayed.

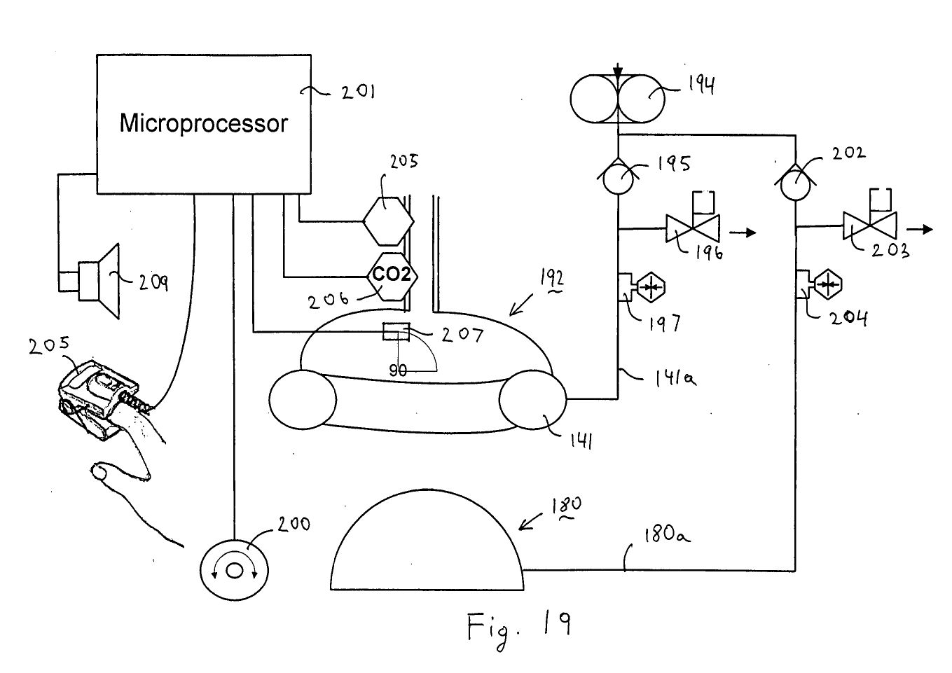

FIGS. 18 and 19 illustrate further optional features that may be provided in the medical emergency kit.

One feature illustrated in FIGS. 18 and 19 is a non-invasive device capable of performing an automatic optimization of the degree of hyperextension (head tilt) of the patient in order to facilitate minimum airway resistance during patient's ventilation. This device is particularly important for ex-hospital use, e.g., during ambulance transportation of the patient, and also for in-hospital use, e.g., for patients undergoing anesthesia or non-invasive ventilation. Optimization of the head tilt is performed automatically by a gradual change of pressure at the neck rest 180 and a gradual change of the tension of strap 165a. As can be shown at FIG. 12, inflation of neck rest 180 will raise the neck, while tensioning strap 165 will decline the forehead. The pressure gradients at patient's ventilation are analyzed by pressure sensor 205.

Thus, as shown in FIG. 18, inelastic strap 165b of the neck rest 180, attachable to arm 142c of the face mask assembly 140 to overlie the patient's forehead (as shown in FIG. 12), is provided with a tensioning drum 200 such that rotation of the drum in one direction tensions the strap 165b and thereby increases the tilt angle of the patient's head. Tensioning drum 200 is controlled by a microprocessor 201 (FIG. 19). Microprocessor 201 also controls the pressure of neck rest 180 and the degree of neck rise. During the gradual change of tension of strap 165b and neck rest pressure, patient's ventilation is performed. The ventilation pressure gradient is detected by pressure sensor 205, and the optimal value (minimum pressure gradient) for both parameters is selected at a specific mask position as indicated by inclination sensor 207.

FIG. 19 illustrates the non-invasive medical kit as including an inflatable neck rest 180, which is inflated by compressor 194 used for inflating the seal 141 of the face mask assembly 192. Accordingly, FIG. 19 includes an additional one-way valve 202 for inflating neck rest 180, an exhaust valve 203 for draining the neck rest, and a pressure sensor 204 for indicating the pressure in the neck rest.

As also seen in FIG. 19, the face mask assembly 192 includes a flow and pressure sensor 205, such as a thermistor, for sensing the spontaneous ventilation flow and pressure of the oxygen inhalations into the face mask. Sensor 206 monitors the carbon dioxide concentration in the exhaled gas. Face mask assembly 192 further includes an inclination sensor 207 for sensing the inclination of the face mask needed for the optimization procedure described above. The outputs of the three sensors 205, 206 and 207 are fed into microprocessor 201.

In the example illustrated in FIG. 19, the heart pulse of the patient is detected by a finger probe ECG 205 which information is also inputted into microprocessor 201.

Microprocessor 201 preferably also includes pre-recorded instructions intended for guiding the operator with respect to the use of the kit. Such pre-recorded instructions are outputted via a speaker 209.

Today, it is recommended that a team of two rescuers perform the basic CPR operation before the arrival of a professional team to the scene. When using the above-described non-invasive medical kits of FIGS. 9-19, only one rescuer may be sufficient.

The standard resuscitation procedures include: electrical shock (defibrillation), mouth to mouth ventilation, and manual chest-compressions. In the above-described kits, the defibrillator is included together with automatic ventilation. Also, due to the capability to produce Negative End Respiratory Pressures (NEEP) by respiratory pump device 100, the system is capable of enhancing or producing some degree of cardiac blood flow.

In the “Automatic chest compression” process described by the system shown in FIGS. 9 and 12, the bystander only needs to connect the mask, the defibrillator and the pulse-oximeter; whereas all the other operations are done automatically.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims. All publications, patents, patent applications and sequences identified by their accession numbers mentioned in this specification are herein incorporated in their entirety by reference into the specification, to the same extent as if each individual publication, patent, patent application or sequence identified by their accession number was specifically and individually indicated to be incorporated herein by reference. In addition, citation or identification of any reference in this application shall not be construed as an admission that such reference is available as prior art to the present invention.

1. An emergency medical kit for rendering emergency medical treatment to a patient, comprising:
a housing;
a pressurized-oxygen container within said housing;
a face mask within said housing and removable therefrom for application to the face of a patient requiring emergency medical treatment;
and a respiratory pump within said housing; said respiratory pump being connectable to said pressurized-oxygen container so as to be driven thereby and also being connectable to said face mask to supply oxygen to said face mask for inhalation by the patient and to discharge exhalations of the patient to the atmosphere.

2. The kit according to claim 1, wherein said respiratory pump includes:
a pump housing having first and second end walls at opposite ends thereof;
a partition wall between said end walls;
a first piston movable between said first end wall and said partition wall and defining a first chamber with said first end wall, and a second chamber with said partition wall;
a second piston movable between said partition wall and said second end wall, and defining a third chamber with said partition wall, and a fourth chamber with said second end wall; and
a stem coupling said first and second pistons for reciprocation together.

3. The kit according to claim 1, wherein said kit further comprises a valve assembly connectable to said pressurized-oxygen container for utilizing the energy of the pressurized oxygen therein to reciprocate said pistons within their respective chambers.

4. The kit according to claim 2, wherein: said pressurized-oxygen container is connected to said first chamber by a first tube; said first chamber is connected to said face mask by a second tube; and said face mask is connected to said third chamber by a third tube.

5. The kit according to claim 4, wherein: the ends of said second and third tubes adjacent to said face mask are coaxial; said second tube includes a one-way valve permitting only inhalation via said face mask; and said third tube includes a one-way valve permitting only exhalations via said face mask.

6. The kit according to claim 4, wherein said valve assembly includes:
a valve control member defining a first valve connecting said pressurized-oxygen container to said first chamber, a second valve connecting said first chamber to said face mask, and a third valve connecting said face mask to said third chamber,
and a drive for reciprocating said valve control member such that during reciprocations thereof in one direction, said first and third valves are opened and said second valve is closed; and during reciprocations in the opposite direction, said first and third valves are closed, and said second valve is opened.

7. The kit according to claim 6, wherein said valve control member is a valve stem, and said drive includes a motor for reciprocating said valve stem.

8. The kit according to claim 3, wherein said second chamber is vented to the atmosphere, and said second piston includes a one-way valve permitting fluid flow therethrough from said fourth chamber during reciprocations of said second piston in one direction, and blocking fluid flow therethrough into said fourth chamber during reciprocations of said second piston in the opposite direction.

9. The kit according to claim 3, wherein said pump further includes a spring acting on said pistons for producing the reciprocations in said opposite direction.

10. The kit according to claim 1, wherein said face mask includes a plate configured to cover the nose and mouth of the patient receiving the mask, and an inflatable seal around the circumference of said plate and engageable with the face of the patient for sealing the interior of the mask with respect to the outside atmosphere; said inflatable seal including a deformable fluid compartment and a pressure sensor sensing the pressure therein; said mask further including an indicator controlled by said pressure sensor for indicating, according to the sensed pressure, whether the face mask is properly sealed with respect to the face of the patient.

11. The kit according to claim 1, wherein the kit further comprises a neck rest removably disposed within said housing; said neck rest being configured for supporting the neck of a patient in need of medical treatment, when the patient is in a reclining position, to facilitate application of the face mask to the patient, the delivery of oxygen for inhalation by the patient, and the discharge to the atmosphere of the exhalations of the patient, with minimum flow resistance.

12. The kit according to claim 11, wherein said neck rest comprises a pair of spaced, parallel side walls engageable at one of their ends with a horizontal surface receiving the patient in a reclining position, and an upper wall of concave configuration for supporting the neck of the patient when received in said reclining position.

13. The kit according to claim 1, wherein said kit further comprises a pulse-oximeter detector probe for application to the patient to detect the patient's pulse.

14. The kit according to claim 13, wherein said face mask is connectable to said respiratory pump by a feed tube; and wherein said pulse-oximeter detector includes an electrical conductor carried by said feed tube for connection to an electrical control system.

15. The kit according to claim 1, wherein said housing further includes a plurality of electrodes for application to the patient for administering electrical pulse therapy to the patient.

16. The kit according to claim 15, wherein said plurality of electrodes include electrical conductors carried by said feed tube for connection to an electrical control system.

17. The kit according to claim 1, wherein said kit further comprises a telephone communication system for receiving remote instructions via the telephone, a GPS locator system for determining the location of the patient being treated, a data logging system for logging data inputted or generated during the operation of the system, a visual display for displaying data inputted or generated during the operation of the system, and/or an audio instruction and alarm system for receiving instructional information and/or for operating an alarm under predetermined conditions.

18. The kit according to claim 1, wherein said housing further includes a built-in testing system, and a test indicator for indicating whether or not the system is operating properly.

19. The kit according to claim 1, wherein said kit further comprises a suction tube insertable into the mouth of a patient and connectable to said respiratory pump for drawing out fluids from the patient's mouth.

20. The kit according to claim 1, wherein said kit further comprises an inflatable neck rest.

21. The kit according to claim 20, wherein said kit further comprises a manual pump and/or a gas-discharge cartridge for inflating said inflatable neck rest.

22. The kit according to claim 1, wherein said kit further comprises a neck rest having a plurality of inelastic straps, and said face mask includes a plurality of strap connectors, one connectable to each of said straps, for securely mounting the face mask to the patient when the patient's head is placed on said neck rest.

23. The kit according to claim 22, wherein said face mask further includes a chin alignment bar engageable with the undersurface of the patient?s chin for-aligning the lower end of the face mask with the patient's chin.

24. The kit according to claim 22, wherein said strap connectors are carried on three arms projecting in a three legs-formation from said face mask such that two of said arms project laterally on opposite sides of the face mask so as to be aligned with the opposite sides of the patient's face when the mask is applied to the patient's face, and the third of said arms projects from an end of the face mask to face the patient's forehead when the mask is applied to the patient's face.

25. The kit according to claim 24, wherein the strap on said neck rest connectable to said third arm of the face mask includes a tensioning device for changing the tension of the strap connectable to said third arm.

26. An emergency medical kit for rendering emergency medical treatment to a patient, comprising:
a container;
a face mask within said container and removable therefrom for application to the face of a patient requiring emergency medical treatment;
and a neck rest within said container and removable therefrom; said neck rest being configured for supporting the neck of a patient in need of medical treatment.

27. The kit according to claim 26, wherein said neck rest is inflatable from a non-inflated condition for compact storage within said container to an inflated condition for use in supporting the neck of a patient.

28. The kit according to claim 27, wherein said kit further comprises a manual pump and/or a gas-discharge cartridge for inflating said inflatable neck rest.

29. The kit according to claim 26, wherein said face mask includes an inflatable seal for sealing the face mask to the patient's face when applied thereto.

30. The kit according to claim 29, wherein said face mask further includes a pressure sensor sensing the pressure within said inflatable seal, and an indicator controlled by said pressure sensor.

31. The kit according to claim 29, wherein said kit further comprises a manual pump and/or a gas-discharge cartridge for inflating said inflatable seal of the face mask.

32. The kit according to claim 26, wherein said neck rest includes a plurality of inelastic straps, and said face mask further includes a plurality of strap connectors, one connectable to each of said straps, for securely mounting the face mask to the patient when the patient's head is placed on said neck rest.

33. The kit according to claim 32, wherein said face mask further includes a chin alignment bar engageable with the undersurface of the patient's chin for aligning the lower end of the face mask with the patient's chin.

34. The kit according to claim 32, wherein said strap connectors are carried on three arms projecting in a tree lags formation from said face mask such that two of said arms project laterally on opposite sides of the face mask so as to be aligned with the opposite sides of the patient's face when the mask is applied to the patient's face, and the third of said arms projects from an end of the face mask to face the patient's forehead when the mask is applied to the patient's face.

35. The kit according to claim 34, wherein the inelastic strap on said neck rest connectable to said third arm of the face mask includes a tensioning device for changing the tension of the strap connectable to said third arm.

36. The kit according to claim 26, wherein said container is a plastic bag.

37. A respiratory pump connectable to a source of pressurized oxygen so as to be driven thereby, said respiratory pump comprising:
a pump housing having first and second end walls at opposite ends thereof;
a partition wall between said end walls;
a first piston movable between said first end wall and said partition wall and defining a first chamber with said first end wall, and a second chamber with said partition wall;
a second piston movable between said partition wall and said second end wall, and defining a third chamber with said partition wall, and a fourth chamber with said second end wall;
a stem coupling said first and second pistons for reciprocation together;
and a valve assembly connectable to said source of pressurized-oxygen for utilizing the energy thereof to reciprocate said pistons within their respective chambers.

38. The respiratory pump according to claim 37, wherein said first chamber is connectable to a source of pressurized oxygen by a first tube; said first chamber is also connectable to a face mask by a second tube for supplying oxygen for inhalation; and said third chamber is connectable to said face mask by a third tube for discharging exhalations.

39. The respiratory pump according to claim 38, wherein: the ends of said second and third tubes adjacent to said face mask are coaxial; said second tube includes a one-way valve permitting only inhalation via said face mask; and said third tube includes a one-way valve permitting only exhalations via said face mask.

40. The respiratory pump according to claim 38, wherein said valve assembly includes:
a valve control member defining a first valve connecting said pressurized-oxygen container to said first chamber, a second valve connecting said first chamber to said face mask, and a third valve connecting said face mask to said third chamber;
and a drive for reciprocating said valve control member
such that during reciprocations thereof in one direction, said first and third valves are opened and said second valve is closed; and during reciprocations in the opposite direction, said first and third valves are closed, and said second valve is opened.

41. The respiratory pump according to claim 38, wherein said valve control member is a valve stem, and said drive includes a motor for reciprocating said valve stem.

42. The respiratory pump according to claim 38, wherein said second chamber is vented to the atmosphere, and said second piston includes a one-way valve permitting fluid flow therethrough from said fourth chamber during reciprocations of said second piston in one direction, and blocking fluid flow therethrough into said fourth chamber during reciprocations of said second piston in the opposite direction.

43. The respiratory pump according to claim 42, wherein said pump housing further includes a spring acting on said pistons for producing the reciprocations in said opposite direction.

44. The respiratory pump according to claim 38, wherein said first piston and said first and second chambers are of smaller cross-sectional area than said second piston and said third and fourth chambers.

45. A face mask for use by a patient; comprising: a plate configured to cover the nose and mouth of the patient, and an inflatable seal around the circumference of said plate and engageable with the face of the patient for sealing the interior of the mask with respect to the outside atmosphere; said inflatable seal including a deformable fluid compartment, and a pressure sensor sensing the pressure therein; said mask further including an indicator controlled by said pressure sensor for indicating, according to the sensed pressure, whether the face mask is properly attached with respect to the face of the patient.

46. The face mask according to claim 45, wherein said face mask further includes a maximum positive-pressure release valve, and a maximum negative-pressure release valve, to prevent the pressure within the mask from exceeding predetermined positive and negative limits.

47. The face mask according to claim 45, wherein said face mask further comprises:
a feed tube for supplying oxygen for inhalation by the patient and/or for discharging exhalations of the patient to the atmosphere;
and a pulse-oximeter detector for application to the patient to detect the patient's pulse; said pulse-oximeter detector including an electrical conductor carried by said feed tube for connection to an electrical control system.

48. The face mask according to claim 45, wherein said face mask further comprises:
a feed tube for supplying oxygen for inhalation by the patient and/or for discharging exhalations of the patient to the atmosphere;
and a plurality of electrodes for application to the patient for administering electrical pulse energy to the patient; said plurality of electrodes including electrical conductors carried by said feed tube for connection to an electrical control system.

49. A face mask for use by a patient, comprising:
a plate configured to cover the nose and mouth of the patient;
a flexible seal around the circumference of said plate and engageable with the face of the patient for sealing the interior of the mask with respect to the outside atmosphere;
a feed tube for supplying oxygen for inhalation by the patient and/or for discharging exhalations of the patient to the atmosphere;
and a pulse-oximeter detector for application to the patient to detect the patient's pulse; said pulse-oximeter detector including an electrical conductor carried by said feed tube for connection to an electrical control system.

50. The face mask according to claim 49, wherein said face mask further comprises a plurality of electrodes for application to the patient for administering electrical pulse therapy to the patient; said plurality of electrodes including electrical conductors also carried by said feed tube for connection to an electrical power supply.

51. The face mask according to claim 50, wherein said flexible seal includes a deformable fluid compartment; and wherein said face mask further includes a pressure sensor sensing the pressure therein, and an indicator controlled by said pressure sensor for indicating, according to the sensed pressure, whether the face mask is properly seal with respect to the face of the patient.
